(19) United States
(12) Patent Application Publication
(10) Pub. No.: US 2026/0250313 A1
(43) Pub. Date: Aug. 27, 2026

(54) USE OF KOSMOTROPES TO ENHANCE YIELD OF AN AFFINITY CHROMATOGRAPHY PURIFICATION STEP

(71) Applicant: Genentech, Inc., South San Francisco, CA (US)

(72) Inventors: Ambrose J. WILLIAMS, REDWOOD CITY, CA (US); Nishit C. SITAPARA, South San Francisco, CA (US); Minjeong CHA, Foster City, CA (US)

(73) Assignee: Genentech, Inc., South San Francisco, CA (US)

(21) Appl. No.: 18/874,894

(22) PCT Filed: Jun. 15, 2023
(86) PCT No.: PCT/US2023/068495

§ 371 Date: Dec. 13, 2024

Related U.S. Application Data
(60) Provisional application No. 63/353,512, filed on Jun. 17, 2022.

(51) Int. Cl. C07K 1/22; C07K 16/00

(52) U.S. Cl. CPC C07K 1/22; C07K 16/00; C07K 2317/21; C07K 2317/24; C07K 2317/31; C07K 2317/55; C07K 2317/56

(57) ABSTRACT

The present disclosure provides methods and kits for purifying an antigen binding protein comprising a VL domain using a Protein L chromatography material which includes kosmotrope salts in the buffer background.

### CROSS-REFERENCE TO RELATED APPLICATIONS

This application claims priority from U.S. provisional application No. 63/353,512, filed Jun. 17, 2022, entitled “USE OF KOSMOTROPES TO ENHANCE YIELD OF AN AFFINITY CHROMATOGRAPHY PURIFICATION STEP,” the contents of which are incorporated by reference in their entirety.

### TECHNICAL FIELD

The present disclosure relates generally to methods for purifying an antigen binding protein comprising a VL region, comprising binding the polypeptide to Protein L chromatography material, washing the chromatography material with a buffer comprising a kosmotrope, and eluting the antigen binding protein at low pH.

### BACKGROUND

Protein L is an affinity ligand that can be used in the chromatographic purification of antibodies as well some antibody fragments such as Fabs, scFvs and VHH. All three of which lack an Fc domain and therefore not compatible with Protein A. Protein L has affinity for the VL domain of kappa light chains, the same domain where three complementarity determining regions (CDRs) are located, which may alter binding efficacy in a manner specific to molecule sequence. Protein L chromatography resins bear Protein L ligands on the surface of the bead, and are usually operated in conditions similar to Protein A chromatography (equilibration, loading of highly-impure feedstock, wash, followed by elution to recover purified protein). What is needed is an improved method of Protein L chromatography.

All references cited herein, including patent applications and publications, are incorporated by reference in their entirety.

### BRIEF SUMMARY

Provided herein is a method for purifying an antigen binding protein comprising a VL domain, the method comprising: a) binding the antigen binding protein to a Protein L chromatography material, b) washing the Protein L chromatography material with a wash buffer, wherein the wash buffer comprises an equilibration buffer and a kosmotrope, and c) eluting the antigen binding protein from the Protein L chromatography material with an elution buffer.

Also provided herein is a method for improving Protein L purification of an antigen binding protein comprising a VL domain, the method comprising: a) binding the antigen binding protein to a Protein L chromatography material, b) washing the Protein L chromatography material with a wash buffer, wherein the wash buffer comprises an equilibration buffer and a kosmotrope, and c) eluting the antigen binding protein from the Protein L chromatography material with an elution buffer.

In some embodiments, the yield and/or purity of the antigen binding protein is increased compared to Protein L chromatography where the wash buffer does not comprise a kosmotrope.

In some of any of such embodiments, the equilibration buffer comprises Tris, MES, MOPS, or EDTA. In some of any of such embodiments, the equilibration buffer comprises Tris and NaCl. In some of any of such embodiments, the Tris is present in the equilibration buffer at a concentration of about 10 mM to about 50 mM, or about 25 mM. In some of any of such embodiments, the NaCl is present in the equilibration buffer at a concentration of about 10 mM to about 50 mM, or about 25 mM. In some of any of such embodiments, the equilibration buffer has a pH of about 4 to about 10. In some of any of such embodiments, the equilibration buffer has a pH of about 6.5 to about 8.5, or about 7.7. In some of any of such embodiments, the equilibration buffer has a pH of about 7 to about 8.

In some of any of such embodiments, the wash buffer has a pH of about 4 to about 10. In some of any of such embodiments, the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5. In some of any of such embodiments, the wash buffer has a pH of about 4, about 4.5, about 5, about 5.5, about 6, about 6.5, about 7, about 7.5, about 8, about 8.5, about 9, about 9.5, or about 10. In some of any of such embodiments, the wash buffer has a pH of about 7.0, about 7.1, about 7.2, about 7.3, about 7.4, about 7.5, about 7.6, about 7.7, about 7.8, about 7.9, or about 8.0.

In some of any of such embodiments, the antigen binding protein is loaded onto the protein L chromatography material in step a) as a host cell culture supernatant.

In some of any of such embodiments, the antigen binding protein is loaded onto the protein L chromatography material in step a) as a purified polypeptide solution.

In some of any of such embodiments, the antigen binding protein is loaded onto the protein L chromatography material in step a) as a mixture from a prior purification step.

In some of any of such embodiments, the antigen binding protein is formulated in equilibration buffer prior to loading onto the protein L chromatography material in step a).

In some of any of such embodiments, the antigen binding protein is formulated in equilibration buffer and the kosmotrope prior to loading onto the protein L chromatography material in step a).

In some of any of such embodiments, the kosmotrope is a phosphate salt or a sulfate salt. In some of any of such embodiments, the kosmotrope is potassium phosphate, sodium sulfate, or ammonium sulfate.

In some of any of such embodiments, the concentration of the kosmotrope in the equilibration buffer or the wash buffer is about 100 mM to about 800 mM. In some of any of such embodiments, the concentration of the kosmotrope in the equilibration buffer and the wash buffer is about 100 mM to about 800 mM. In some of any of such embodiments, the concentration of the kosmotrope in the equilibration buffer or the wash buffer is about 120 mM to about 600 mM. In some of any of such embodiments, the concentration of the kosmotrope in the equilibration buffer or the wash buffer is about 120 mM, about 240 mM, about 360 mM, about 480 mM, or about 600 mM. In some of any of such embodiments, the concentration of the kosmotrope in the equilibration buffer and/or the wash buffer is about 100 mM to about 800 mM, about 100 mM to about 700 mM, about 100 mM to about 600 mM, about 100 mM to about 500 mM, about 200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM. In some of any of such embodiments, the concentration of the kosmotrope in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM.

In some of any of such embodiments, the kosmotrope is sodium sulfate. In some of any of such embodiments, the kosmotrope is potassium phosphate. In some of any of such embodiments, the kosmotrope is ammonium sulfate.

In some of any of such embodiments, the elution buffer has a lower pH than the equilibration buffer. In some of any of such embodiments, the elution buffer has a pH of about 2.0 to about 4.0, or about 2.5 to about 3.0. In some of any of such embodiments, the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3. In some of any of such embodiments, the elution buffer has a pH of about 2.7, about 2.75, about 2.8, about 2.85, about 2.9, or about 2.95. In some of any of such embodiments, the elution buffer has a higher pH than the equilibration buffer. In some of any of such embodiments, the elution buffer has a pH of about 10.0 to about 12.0.

In some of any of such embodiments, the elution buffer comprises acetic acid. In some of any of such embodiments, the elution buffer comprises about 50 mM acetic acid to about 250 mM acetic acid.

In some of any of such embodiments, the elution buffer comprises about 150 mM acetic acid at a pH of about 2.8.

In some of any of such embodiments, the elution buffer comprises sodium acetate, citrate, or glycine.

In some of any of such embodiments, the antigen binding protein binds poorly to Protein L. In some of any of such embodiments, the antigen binding protein binds poorly to Protein L as compared to a reference antigen binding protein that is monoclonal antibody G6-31. In some of any of such embodiments, the antigen binding protein binds weaker to Protein L than a reference antigen binding protein that is monoclonal antibody G6-31.

In some of any of such embodiments, the antigen binding protein is an antibody or immunoadhesin or a fragment thereof. In some of any of such embodiments, the antigen binding protein is a monoclonal antibody. In some of any of such embodiments, the antigen binding protein is a human antibody, a chimeric antibody or a humanized antibody. In some of any of such embodiments, the antibody is an antibody fragment selected from a Fab, Fab′-SH, Fv, scFv, and (Fab′)2 fragment. In some of any of such embodiments, the antibody fragment is a Fab. In some of any of such embodiments, the Fab comprises a binding site for binding with Protein L that lacks a hydrophobic patch or has a weak hydrophobic patch. In some of any of such embodiments, the binding site for binding with Protein L lacks a hydrophobic patch. In some of any of such embodiments, the binding site for binding with Protein L has a weak hydrophobic patch. In some of any of such embodiments, the binding site for binding with Protein L has a weak hydrophobic patch as compared to the binding site for binding with Protein L of a reference antigen binding protein that is monoclonal antibody G6-31. In some of any of such embodiments, the binding site for binding with Protein L comprises a hydrophilic patch.

In some of any of such embodiments, the binding affinity (EC50) and/or the dissociation constant of the antigen binding protein to Protein L has a value that is higher than the value of the binding affinity (EC50) and/or the dissociation constant of a reference antigen binding protein that is monoclonal antibody G6-31 to Protein L. In some of any of such embodiments, the binding affinity (EC50) and/or the dissociation constant of the antigen binding protein to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.

In some of any of such embodiments, the antibody does not contain an Fc domain.

In some of any of such embodiments, the antibody is a multispecific antibody. In some of any of such embodiments, the multispecific antibody comprises the VL domain and a second VL domain, and wherein the VL domain is a kappa (κ) VL. In some of any of such embodiments, the VL domain is a κ2 VL. In some of any of such embodiments, the VL domain binds poorly to Protein L. In some of any of such embodiments, the VL domain binds poorly to Protein L and the second VL domain does not bind to Protein L. In some of any of such embodiments, the second VL domain is a lambda (λ) VL. In some of any of such embodiments, the VL domain is a 2 subtype VL or a κ VL. In some of any of such embodiments, the VL domain binds poorly to Protein L. In some of any of such embodiments, the VL domain binds poorly to Protein L as compared to the binding of the VL domain of a reference antigen binding protein that is monoclonal antibody G6-31 to Protein L. In some of any of such embodiments, the VL domain is a κ VL and comprises a modification that weakens binding of the VL domain to Protein L. In some of any of such embodiments, the modification comprises one or more amino acid substitutions in the VL domain. In some of any of such embodiments, the VL domain is a κ2 VL.

In some of any of such embodiments, the antigen binding protein comprises a binding site for binding with Protein L that lacks a hydrophobic patch or has a weak hydrophobic patch. In some embodiments, the binding site for binding with Protein L lacks a hydrophobic patch. In some embodiments, the binding site for binding with Protein L has a weak hydrophobic patch. In some of any of such embodiments, the binding site for binding with Protein L has a weak hydrophobic patch as compared to the binding site for binding with Protein L of a reference antigen binding protein that is monoclonal antibody G6-31. In some of any of such embodiments, the binding site for binding with Protein L comprises a hydrophilic patch.

In some of any of such embodiments, the protein L chromatography material is a Pierce™ Protein L chromatography cartridge, a Capto™ L chromatography, HiTrap® Protein L chromatography, a KanCap™ L chromatography, a TOYOPEARL® AF-rProtein L-650F chromatography, or a MabSelect™ VL chromatography.

Also provided herein is a composition comprising an antigen binding protein purified by a method comprising any of the methods described herein. In some embodiments, the composition comprises one or more pharmaceutical excipients.

Also provided herein is a kit for purifying an antigen binding protein comprising a VL domain, wherein the kit comprises a Protein L chromatography material and a kosmotrope.

In some embodiments, the kit further comprises an equilibration buffer. In some embodiments, the equilibration buffer comprises Tris, MES, MOPS, or EDTA. In some of any of such embodiments, the equilibration buffer comprises Tris and NaCl. In some of any of such embodiments, the Tris is present in the equilibration buffer at a concentration of about 10 mM to about 50 mM or about 25 mM. In some of any of such embodiments, the NaCl is present in the equilibration buffer at a concentration of about 10 mM to about 50 mM or about 25 mM.

In some of any of such embodiments, the equilibration buffer has a pH of about 4 to about 10. In some of any of such embodiments, the equilibration buffer has a pH of about 6.5 to about 8.5, or about 7.7. In some of any of such embodiments, the equilibration buffer has a pH of about 7 to about 8.

In some of any of such embodiments, the kit further comprises a wash buffer. In some embodiments, the wash buffer has a pH of about 4 to about 10. In some of any of such embodiments, the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5. In some of any of such embodiments, the wash buffer has a pH of about 4, about 4.5, about 5, about 5.5, about 6, about 6.5, about 7, about 7.5, about 8, about 8.5, about 9, about 9.5, or about 10. In some of any of such embodiments, the wash buffer has a pH of about 7.0, about 7.1, about 7.2, about 7.3, about 7.4, about 7.5, about 7.6, about 7.7, about 7.8, about 7.9, or about 8.0.

In some of any of such embodiments, the wash buffer comprises the equilibration buffer and the kosmotrope. In some of any of such embodiments, the equilibration buffer further comprises the kosmotrope.

In some of any of such embodiments, the kosmotrope is a phosphate salt or a sulfate salt. In some of any of such embodiments, the kosmotrope is potassium phosphate, sodium sulfate, or ammonium sulfate. In some of any of such embodiments, the concentration of the kosmotrope in the equilibration buffer or the wash buffer is about 100 mM to about 800 mM. In some of any of such embodiments, the concentration of the kosmotrope in the equilibration buffer and the wash buffer is about 100 mM to about 800 mM. In some of any of such embodiments, the concentration of the kosmotrope in the equilibration buffer or the wash buffer is about 120 mM to about 600 mM. In some of any of such embodiments, the concentration of the kosmotrope in the equilibration buffer or the wash buffer is about 120 mM, about 240 mM, about 360 mM, about 480 mM, or about 600 mM. In some of any of such embodiments, the concentration of the kosmotrope in the equilibration buffer and/or the wash buffer is about 100 mM to about 800 mM, about 100 mM to about 700 mM, about 100 mM to about 600 mM, about 100 mM to about 500 mM, about 200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM. In some of any of such embodiments, the concentration of the kosmotrope in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM.

In some of any of such embodiments, the kosmotrope is sodium sulfate. In some of any of such embodiments, the kosmotrope is potassium phosphate. In some of any of such embodiments, the kosmotrope is ammonium sulfate.

In some of any of such embodiments, the kit further comprises an elution buffer. In some embodiments, the elution buffer has a lower pH than the equilibration buffer. In some of any of such embodiments, the elution buffer has a pH of about 2.0 to about 4.0 or about 2.5 to about 3.0. In some of any of such embodiments, the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3. In some of any of such embodiments, the elution buffer has a pH of about 2.7, about 2.75, about 2.8, about 2.85, about 2.9, or about 2.95.

In some embodiments, the elution buffer has a higher pH than the equilibration buffer. In some of any of such embodiments, the elution buffer has a pH of about 10.0 to about 12.0.

In some of any of such embodiments, the elution buffer comprises acetic acid. In some embodiments, the elution buffer comprises about 50 mM acetic acid to about 250 mM acetic acid. In some of any of such embodiments, the elution buffer comprises about 150 mM acetic acid at a pH of about 2.8. In some of any of such embodiments, the elution buffer comprises sodium acetate, citrate, or glycine.

In some of any of such embodiments, the kit is for use in purifying an antibody or immunoadhesin or a fragment thereof. In some of any of such embodiments, the antigen binding protein is a monoclonal antibody. In some of any of such embodiments, the antigen binding protein is a human antibody, a chimeric antibody or a humanized antibody. In some of any of such embodiments, the antibody is an antibody fragment selected from a Fab, Fab′-SH, Fv, scFv, and (Fab′)2 fragment. In some of any of such embodiments, the antibody fragment is a Fab.

In some embodiments, the Fab comprises a binding site for binding with Protein L that lacks a hydrophobic patch or has a weak hydrophobic patch. In some embodiments, the binding site for binding with Protein L lacks a hydrophobic patch. In some embodiments, the binding site for binding with Protein L has a weak hydrophobic patch. In some of any of such embodiments, the binding site for binding with Protein L has a weak hydrophobic patch as compared to the binding site for binding with Protein L of a reference antigen binding protein that is monoclonal antibody G6-31. In some of any of such embodiments, the binding site for binding with Protein L comprises a hydrophilic patch.

In some of any of such embodiments, the binding affinity (EC50) and/or the dissociation constant of the antigen binding protein to Protein L has a value that is higher than the value of the binding affinity (EC50) and/or the dissociation constant of a reference antigen binding protein that is monoclonal antibody G6-31 to Protein L. In some of any of such embodiments, the binding affinity (EC50) and/or the dissociation constant of the antigen binding protein to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.

In some of any of such embodiments, the antibody does not contain an Fc domain.

In some of any of such embodiments, the antibody is a multispecific antibody. In some embodiments, the multispecific antibody comprises a VL domain and a second VL domain, and wherein the VL domain is a kappa (κ) VL. In some of any of such embodiments, the VL domain is a κ2 VL. In some of any of such embodiments, the VL domain binds poorly to Protein L. In some of any of such embodiments, the VL domain binds poorly to Protein L and the second VL domain does not bind to Protein L. In some of any of such embodiments, the second VL domain is a lambda (λ) VL. In some of any of such embodiments, the antibody comprises a VL domain that is a λ subtype VL or a κ VL. In some of any of such embodiments, the VL domain binds poorly to Protein L. In some of any of such embodiments, the VL domain of the antigen binding protein binds poorly to Protein L as compared to a reference antigen binding protein that is monoclonal antibody G6-31. In some of any of such embodiments, the VL domain of the antigen binding protein binds weaker to Protein L than a reference antigen binding protein that is monoclonal antibody G6-31.

In some of any of such embodiments, the VL domain is a κ VL and comprises a modification that weakens binding of the VL domain to Protein L. In some embodiments, the modification comprises one or more amino acid substitutions in the VL domain. In some of any of such embodiments, the VL domain is a κ2 VL. In some of any of such embodiments, the antigen binding protein comprises a binding site for binding with Protein L that lacks a hydrophobic patch or has a weak hydrophobic patch. In some embodiments, the binding site for binding with Protein L lacks a hydrophobic patch. In some embodiments, the binding site for binding with Protein L has a weak hydrophobic patch. In some of any of such embodiments, the binding site for binding with Protein L has a weak hydrophobic patch as compared to the binding site for binding with Protein L of a reference antigen binding protein that is monoclonal antibody G6-31. In some of any of such embodiments, the binding site for binding with Protein L comprises a hydrophilic patch.

### BRIEF DESCRIPTION OF THE DRAWINGS

FIG. 1 shows a chromatogram from purification of Fab 1 (a Fab antibody fragment) on a Protein L resin. UV absorbance is suppressed during a phosphate-containing wash phase with high conductivity, but resumes after the end of that phase.

FIG. 2 shows UV Absorbance signal overlays across six chromatography runs using wash buffers comprising different amounts of sodium sulfate (0 mM, 120 mM, 240 mM, 360 mM, 480 mM and 600 mM). Protein exiting the column is detected by UV trace (intensity in milli-absorbance units “mAU” on Y axis). In each run, the column is equilibrated prior to injection at volume=0 mL (on X-axis). Load phase produces high UV signal due to high burden of host impurities in feedstock (harvested cell culture fluid). At the mark “1.A.1” load phase ends and wash phase begins, proceeding through mark 1.A.2. Elution phase recovers purified protein at mark 1.A.3 at approximately 120 mL volume on X axis.

FIG. 3 shows SEC-HPLC analysis of Fab 1 following the chromatography runs shown in FIG. 2. In addition to the Fab, high molecular weight fragments (HMWF) and light chain (LC) dimers were detected.

FIG. 4 shows UV Absorbance signal overlays across six chromatography runs using wash buffers comprising different amounts of potassium phosphate (0 mM, 120 mM, 240 mM, 360 mM, 480 mM and 600 mM).

FIG. 5 shows SEC-HPLC analysis of Fab 1 following the chromatography runs shown in FIG. 4. In addition to the Fab, high molecular weight fragments (HMWF) and light chain (LC) dimers were detected.

FIG. 6 shows UV Absorbance signal overlays across six chromatography runs using wash buffers comprising different amounts of sodium chloride (0 mM, 120 mM, 240 mM, 360 mM, 480 mM and 600 mM).

FIG. 7 shows SEC-HPLC analysis of Fab 1 following the chromatography runs shown in FIG. 6. In addition to the Fab, high molecular weight fragments (HMWF) and light chain (LC) dimers were detected.

FIG. 8 shows a chromatograph from a protein L chromatography with a bispecific antibody were no sulfate was included in the wash buffer (i.e., a control experiment). The chromatogram shows the UV absorbance signal as well as signals for pH and conductivity. The rise in UV early illustrates protein breaking through during the load phase, and washing off during the wash phase. A modest elution peak is recovered around the 34-minute mark.

FIG. 9 shows a chromatogram from a protein L chromatography with a bispecific antibody where 600 mM Sulfate was present in both load feedstock and the wash buffer. Run chromatogram containing UV absorbance signal as well as signals for pH and conductivity. The rise in UV early illustrates protein breaking through during the load phase, and washing off during the wash phase. A modest elution peak is recovered around the 34-minute mark.

### DETAILED DESCRIPTION

In some aspects, the invention provides methods for purifying an antigen binding protein comprising a VL domain, the method comprising a) binding the antigen binding protein to a Protein L chromatography material, b) washing the Protein L chromatography material with a wash buffer, wherein the wash buffer comprises an equilibration buffer and a kosmotrope (e.g., a sulfate or a phosphate), c) eluting the antigen binding protein from the Protein L chromatography material with an elution buffer. In some aspects, the invention provides methods for improving Protein L purification of an antigen binding protein comprising a VL domain, the method comprising a) binding the antigen binding protein to a Protein L chromatography material, b) washing the Protein L chromatography material with a wash buffer, wherein the wash buffer comprises an equilibration buffer and a kosmotrope (e.g., a sulfate or a phosphate), c) eluting the antigen binding protein from the Protein L chromatography material with an elution buffer. In some embodiments, the yield and/or purity of the antigen binding protein is increased compared to Protein L chromatography where the wash buffer does not comprise a kosmotrope.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

As used herein, a “kosmotrope,” less frequently spelled “cosmotrope,” is a salt which promotes hydrophobic binding due to the way they structure water molecules in solution. Two examples of kosmotropic anions used in chromatography are phosphate and sulfate. Another kosmotrope, ammonium sulfate, is so strongly kosmotropic it is frequently used in salt-cutting (precipitation) of proteins out of solution. In contrast to kosmotropes are the chaotropic salts which disrupt hydrophobic binding; the Hofmeister series is a ranking of different salts along a kosmotrope-vs-chaotrope spectrum according to their properties.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

“Purified” polypeptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity.

The term “antibody” includes full-length antibodies and antigen-binding fragments thereof. In some embodiments, a full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain). In some embodiments, the antibody is a chimeric antibody. In some embodiments, the antibody is a semi-synthetic antibody. In some embodiments, the antibody is a diabody. In some embodiments, the antibody is a humanized antibody. In some embodiments, the antibody is a multispecific antibody, such as a bispecific antibody. In some embodiments, the antibody is linked to a fusion protein. In some embodiments, the antibody is linked to an immunostimulating protein, such as an interleukin. In some embodiments, the antibody is linked to a protein which facilitates the entry across the blood brain barrier.

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)).

The term “multispecific antibodies” as used herein refer to monoclonal antibodies that have binding specificities for at least two different sites, i.e., different epitopes on different antigens or different epitopes on the same antigen. In certain aspects, the multispecific antibody has two binding specificities (bispecific antibody). In certain aspects, the multispecific antibody has three or more binding specificities. Multispecific antibodies may be prepared as full length antibodies or antibody fragments.

The term “semi-synthetic” in reference to an antibody or antibody means that the antibody or antibody has one or more naturally occurring sequences and one or more non-naturally occurring (i.e., synthetic) sequences.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

“Contaminants” refer to materials that are different from the desired polypeptide product. The contaminant includes, without limitation: host cell materials, such as CHO host cell protein (CHOP); leached Protein A; nucleic acid; a variant (e.g., a basic variant or an acidic variant of the desired polypeptide product), fragment, aggregate or derivative of the desired polypeptide (e.g., high molecular weight species (HMWS) or very high molecular weight species (vHMWS) of the desired polypeptide); another polypeptide; endotoxin; viral contaminant; cell culture media component, etc. In some examples, the contaminant may be a host cell protein (HCP) from, for example but not limited to, a bacterial cell such as an E. coli cell, an insect cell, a prokaryotic cell, a eukaryotic cell, a yeast cell, a mammalian cell, an avian cell, a fungal cell.

The term “hydrophobic patch” refers to a portion of an antigen binding protein, e.g., an antibody or immunoadhesin or fragment thereof, such as a Fab, that contains one or more hydrophobic amino acid residues that give it an overall hydrophobic nature. The term “hydrophilic patch” refers to a portion of an antigen binding protein, e.g., an antibody or immunoadhesin or fragment thereof, such as a Fab, that contains one or more hydrophilic amino acid residues that give it an overall hydrophilic nature.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### Use of Kosmotropes in Protein L Chromatography

In some aspects, the invention provides methods for purifying an antigen binding protein comprising a VL domain, the method comprising a) binding the antigen binding protein to a Protein L chromatography material, b) washing the Protein L chromatography material with a wash buffer, wherein the wash buffer comprises an equilibration buffer and a kosmotrope (e.g., a sulfate or a phosphate), c) eluting the antigen binding protein from the Protein L chromatography material with an elution buffer. In some aspects, the invention provides methods for improving Protein L purification of an antigen binding protein comprising a VL domain, the method comprising a) binding the antigen binding protein to a Protein L chromatography material, b) washing the Protein L chromatography material with a wash buffer, wherein the wash buffer comprises an equilibration buffer and a kosmotrope (e.g., a sulfate or a phosphate), c) eluting the antigen binding protein from the Protein L chromatography material with an elution buffer. In some embodiments, the yield and/or purity of the antigen binding protein is increased compared to Protein L chromatography where the wash buffer does not comprise a kosmotrope.

In some embodiments, the equilibration buffer can be any suitable equilibration buffer.

In some embodiments of the invention, the equilibration buffer comprises Tris (i.e., tris(hydroxylmethyl)aminomethane or tromethane). In some embodiments of the inventions, the equilibration buffer comprises NaCl. In some embodiments of the invention, the equilibration buffer comprises Tris and NaCl.

In some embodiments, the Tris is present in the equilibration buffer at a concentration of about 10 mM to about 100 mM. In some embodiments, the Tris is present in the equilibration buffer at a concentration of between about any of 10 mM and 100 mM, 15 mM and 100 mM, 20 mM and 100 mM, 25 mM and 100 mM, 30 mM and 100 mM, 40 mM and 100 mM, 50 mM and 100 mM, 75 mM and 100 mM, 10 mM and 75 mM, 15 mM and 75 mM, 20 mM and 75 mM, 25 mM and 75 mM, 30 mM and 75 mM, 40 mM and 75 mM, 50 mM and 75 mM, 75 mM and 75 mM, 10 mM and 50 mM, 15 mM and 50 mM, 20 mM and 50 mM, 25 mM and 50 mM, 30 mM and 50 mM, 40 mM and 50 mM, 10 mM and 40 mM, 15 mM and 40 mM, 20 mM and 40 mM, 25 mM and 40 mM, 30 mM and 40 mM, 10 mM and 30 mM, 15 mM and 30 mM, 20 mM and 30 mM, 25 mM and 30 mM, 10 mM and 25 mM, 15 mM and 25 mM, 20 mM and 25 mM, 10 mM and 20 mM, 15 mM and 20 mM, or 10 mM and 15 mM. In some embodiments, the Tris is present in the equilibration buffer at a concentration of more than about 10 mM, 15 mM, 20 mM, 25 mM, 30 mM, 40 mM, 50 mM, 75 mM, or 100 mM.

In some embodiments, the Tris is present in the equilibration buffer at a concentration of about 10 mM to about 100 mM. In some embodiments, the Tris is present in the equilibration buffer at a concentration of between about any of 10 mM and 100 mM, 15 mM and 100 mM, 20 mM and 100 mM, 25 mM and 100 mM, 30 mM and 100 mM, 40 mM and 100 mM, 50 mM and 100 mM, 75 mM and 100 mM, 10 mM and 75 mM, 15 mM and 75 mM, 20 mM and 75 mM, 25 mM and 75 mM, 30 mM and 75 mM, 40 mM and 75 mM, 50 mM and 75 mM, 75 mM and 75 mM, 10 mM and 50 mM, 15 mM and 50 mM, 20 mM and 50 mM, 25 mM and 50 mM, 30 mM and 50 mM, 40 mM and 50 mM, 10 mM and 40 mM, 15 mM and 40 mM, 20 mM and 40 mM, 25 mM and 40 mM, 30 mM and 40 mM, 10 mM and 30 mM, 15 mM and 30 mM, 20 mM and 30 mM, 25 mM and 30 mM, 10 mM and 25 mM, 15 mM and 25 mM, 20 mM and 25 mM, 10 mM and 20 mM, 15 mM and 20 mM, or 10 mM and 15 mM. In some embodiments, the Tris is present in the equilibration buffer at a concentration of more than about 10 mM, 15 mM, 20 mM, 25 mM, 30 mM, 40 mM, 50 mM, 75 mM, or 100 mM.

In some embodiments of the invention, the equilibration buffer comprises MES (2-(N-morpholino) ethanesulfonic acid). In some embodiments, the equilibration buffer comprises MES and NaCl. In some embodiments, the equilibration buffer comprises MOPS (3-(N-morpholino) propanesulfonic acid). In some embodiments, the equilibration buffer comprises MOPS and NaCl. In some embodiments, the equilibration buffer comprises EDTA (ethylenediaminetetraacetic acid). In some embodiments, the equilibration buffer comprises a carbonate buffer.

In some embodiments, the NaCl is present in the equilibration buffer at a concentration of about 10 mM to about 100 mM. In some embodiments, the NaCl is present in the equilibration buffer at a concentration of between about any of 10 mM and 100 mM, 15 mM and 100 mM, 20 mM and 100 mM, 25 mM and 100 mM, 30 mM and 100 mM, 40 mM and 100 mM, 50 mM and 100 mM, 75 mM and 100 mM, 10 mM and 75 mM, 15 mM and 75 mM, 20 mM and 75 mM, 25 mM and 75 mM, 30 mM and 75 mM, 40 mM and 75 mM, 50 mM and 75 mM, 75 mM and 75 mM, 10 mM and 50 mM, 15 mM and 50 mM, 20 mM and 50 mM, 25 mM and 50 mM, 30 mM and 50 mM, 40 mM and 50 mM, 10 mM and 40 mM, 15 mM and 40 mM, 20 mM and 40 mM, 25 mM and 40 mM, 30 mM and 40 mM, 10 mM and 30 mM, 15 mM and 30 mM, 20 mM and 30 mM, 25 mM and 30 mM, 10 mM and 25 mM, 15 mM and 25 mM, 20 mM and 25 mM, 10 mM and 20 mM, 15 mM and 20 mM, or 10 mM and 15 mM. In some embodiments, the NaCl is present in the equilibration buffer at a concentration of more than about 10 mM, 15 mM, 20 mM, 25 mM, 30 mM, 40 mM, 50 mM, 75 mM, or 100 mM.

In some embodiments, the Tris is present in the equilibration buffer at a concentration of about 10 mM to about 100 mM and the NaCl is present in the equilibration buffer at a concentration of about 10 mM to about 100 mM. In some embodiments, the Tris is present in the equilibration buffer at a concentration of about 10 mM to about 50 mM and the NaCl is present in the equilibration buffer at a concentration of about 10 mM to about 50 mM. In some embodiments, the Tris is present in the equilibration buffer at a concentration of about 20 mM to about 30 mM and the NaCl is present in the equilibration buffer at a concentration of about 20 mM to about 30 mM. In some embodiments, the Tris is present in the equilibration buffer at a concentration of about 25 mM and the NaCl is present in the equilibration buffer at a concentration of about 25 mM.

In some embodiments, the equilibration buffer has a pH of about 4.0 to about 10.0. In some embodiments, the equilibration buffer has a pH of about 6.0 to about 9.0. In some embodiments, the equilibration buffer has a pH of about 7.0 to about 8.0. In some embodiments, the equilibration buffer has a pH between about any of 6.0 and 9.0, 6.5 and 8.5, or 7.0 and 8.0. In some embodiments, the equilibration buffer has a pH of any of about 6.0, 6.5, 7.0, 7.1, 7.2, 7.3, 7.4, 7.5, 7.6, 7.7, 7.8, 7.9, 8.0, 8.5 or 9.0. In some embodiments, the equilibration buffer has a pH of 7.7 or about 7.7. In some embodiments, the Tris is present in the equilibration buffer at a concentration of about 10 mM to about 100 mM, the NaCl is present in the equilibration buffer at a concentration of about 10 mM to about 100 mM, and the pH of the equilibration buffer is about 6.0 to about 9.0. In some embodiments, the Tris is present in the equilibration buffer at a concentration of about 10 mM to about 50 mM, the NaCl is present in the equilibration buffer at a concentration of about 10 mM to about 50 mM, and the pH of the equilibration buffer is about 6.5 to about 8.5. In some embodiments, the Tris is present in the equilibration buffer at a concentration of about 20 mM to about 30 mM, the NaCl is present in the equilibration buffer at a concentration of about 20 mM to about 30 mM, and the pH of the equilibration buffer is about 7.5 to about 8.0. In some embodiments, the Tris is present in the equilibration buffer at a concentration of about 25 mM, the NaCl is present in the equilibration buffer at a concentration of about 25 mM, and the pH of the equilibration buffer is about 7.7.

In some embodiments, the wash buffer has a pH of about 4 to about 10. In some embodiments, the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5. In some embodiments, the wash buffer has a pH of about 4, about 4.5, about 5, about 5.5, about 6, about 6.5, about 7, about 7.5, about 8, about 8.5, about 9, about 9.5, or about 10. In some embodiments, the wash buffer has a pH of about 7.0, about 7.1, about 7.2, about 7.3, about 7.4, about 7.5, about 7.6, about 7.7, about 7.8, about 7.9, or about 8.0.

In some embodiments, the antigen binding protein is loaded onto the protein L chromatography material in step a) as a host cell culture supernatant (HCCS). In some embodiments, the antigen binding protein is loaded onto the protein L chromatography material in step a) as a purified polypeptide solution. In some embodiments, the antigen binding protein is loaded onto the protein L chromatography material in step a) as a mixture from a prior purification step. The prior purification step can be any step in the purification process or workflow that occurred prior to the loading onto the protein L chromatography material in step a). In some embodiments, the antigen binding protein is formulated in equilibration buffer prior to loading onto the protein L chromatography material in step a). In some embodiments, the kosmotrope prior is added to the HCCS comprising the antigen binding protein prior to loading onto the protein L chromatography material in step a). In some embodiments, the antigen binding protein is formulated in equilibration buffer and the kosmotrope prior to loading onto the protein L chromatography material in step a). In some embodiments, the kosmotrope is a phosphate salt or a sulfate salt. In some embodiments, the kosmotrope is potassium phosphate, sodium sulfate, or ammonium sulfate. In some embodiments, the concentration of the kosmotrope (e.g., potassium phosphate, sodium sulfate, or ammonium sulfate) in the equilibration buffer or the HCCS prior to loading onto the protein L chromatography material in step a) is any concentration of at least about 100 mM that does not result in precipitation of the antigen binding protein out of solution. In some embodiments, the concentration of the kosmotrope (e.g., potassium phosphate, sodium sulfate, or ammonium sulfate) in the equilibration buffer or the HCCS prior to loading onto the protein L chromatography material in step a) is about 100 mM to about 1000 mM. In some embodiments, the concentration of the kosmotrope (e.g., potassium phosphate, sodium sulfate, or ammonium sulfate) in the equilibration buffer or the HCCS prior to loading onto the protein L chromatography material in step a) is between about and of 100 mM and 1000 mM, 120 mM and 1000 mM, 240 mM and 1000 mM, 360 mM and 1000 mM, 480 mM and 1000 mM, 600 mM and 1000 mM, 800 mM and 1000 mM, 100 mM and 800 mM, 120 mM and 800 mM, 240 mM and 800 mM, 360 mM and 800 mM, 480 mM and 800 mM, 600 mM and 800 mM, 100 mM and 600 mM, 120 mM and 600 mM, 240 mM and 600 mM, 360 mM and 600 mM, 480 mM and 600 mM, 100 mM and 480 mM, 120 mM and 480 mM, 240 mM and 480 mM, 360 mM and 480 mM, 100 mM and 360 mM, 120 mM and 360 mM, 240 mM and 360 mM, 100 mM and 240 mM, 120 mM and 240 mM, or 100 mM and 120 mM. In some embodiments, the concentration of the kosmotrope (e.g., potassium phosphate, sodium sulfate, or ammonium sulfate) in the equilibration buffer or the HCCS prior to loading onto the protein L chromatography material in step a) is more than about any of 100 mM, 120 mM, 240 mM, 360 mM, 480 mM, 600 mM, 800 mM or 1000 mM. In some embodiments, the concentration of the kosmotrope in the equilibration buffer is about 100 mM to about 800 mM. In some embodiments, the concentration of the kosmotrope in the equilibration buffer is about 120 mM to about 600 mM.

In some embodiments, the concentration of the kosmotrope (e.g., potassium phosphate, sodium sulfate, or ammonium sulfate) in the equilibration buffer and/or the wash buffer is about 100 mM to about 800 mM, about 100 mM to about 700 mM, about 100 mM to about 600 mM, about 100 mM to about 500 mM, about 200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM. In some embodiments, the concentration of the kosmotrope (e.g., potassium phosphate, sodium sulfate, or ammonium sulfate) in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM. In some embodiments, the kosmotrope is sodium sulfate. In some embodiments, the kosmotrope is potassium phosphate. In some embodiments, the kosmotrope is ammonium sulfate.

In some embodiments, the wash buffer of step b) comprises a kosmotrope. In some embodiments, the kosmotrope in the wash buffer is a phosphate salt or a sulfate salt. In some embodiments, the kosmotrope in the wash buffer is potassium phosphate, sodium sulfate, or ammonium sulfate. In some embodiments, the wash buffer of step b) comprises equilibration buffer and a kosmotrope. In some embodiments, the concentration of the kosmotrope (e.g., potassium phosphate, sodium sulfate, or ammonium sulfate) in the wash buffer of step b) is any concentration of at least about 100 mM that does not result in precipitation of the antigen binding protein out of solution. In some embodiments, the concentration of the kosmotrope (e.g., potassium phosphate, sodium sulfate, or ammonium sulfate) in the wash buffer of step b) is about 100 mM to about 1000 mM. In some embodiments, the concentration of the kosmotrope (e.g., potassium phosphate, sodium sulfate, or ammonium sulfate) in the wash buffer of step b) is between about and of 100 mM and 1000 mM, 120 mM and 1000 mM, 240 mM and 1000 mM, 360 mM and 1000 mM, 480 mM and 1000 mM, 600 mM and 1000 mM, 800 mM and 1000 mM, 100 mM and 800 mM, 120 mM and 800 mM, 240 mM and 800 mM, 360 mM and 800 mM, 480 mM and 800 mM, 600 mM and 800 mM, 100 mM and 600 mM, 120 mM and 600 mM, 240 mM and 600 mM, 360 mM and 600 mM, 480 mM and 600 mM, 100 mM and 480 mM, 120 mM and 480 mM, 240 mM and 480 mM, 360 mM and 480 mM, 100 mM and 360 mM, 120 mM and 360 mM, 240 mM and 360 mM, 100 mM and 240 mM, 120 mM and 240 mM, or 100 mM and 120 mM. In some embodiments, the concentration of the kosmotrope (e.g., potassium phosphate, sodium sulfate, or ammonium sulfate) in the wash buffer of step b) is more than about any of 100 mM, 120 mM, 240 mM, 360 mM, 480 mM, 600 mM, 800 mM or 1000 mM. In some embodiments, the concentration of the kosmotrope in the wash buffer is about 100 mM to about 800 mM. In some embodiments, the concentration of the kosmotrope in the wash buffer is about 120 mM to about 600 mM.

In some embodiments, the concentration of the kosmotrope in the equilibration buffer and the wash buffer is about 100 mM to about 800 mM. In some embodiments, the concentration of the kosmotrope in the equilibration buffer and the wash buffer is about 120 mM to about 600 mM.

In some embodiments, the elution buffer of step c) has a lower pH than the equilibration buffer. In some embodiments, the elution buffer has a pH of about 2.0 to about 5.0. In some embodiments, the elution buffer has a pH of between about any of 2.0 and 5.0, 2.5 and 5.0, 3.0 and 5.0, 3.5 and 5.0, 4.0 and 5.0, 2.0 and 4.0, 2.5 and 4.0, 3.0 and 4.0, 3.5 and 4.0, 2.0 and 3.5, 2.5 and 3.5, 3.0 and 3.5, 2.0 and 3.0, 2.5 and 3.0, or 2.0 and 2.5. In some embodiments, the elution buffer has a pH of any of about 2.0, 2.1, 2.2, 2.3, 2.4, 2.5, 2.6, 2.7, 2.75, 2.8, 2.9, 3.0, 3.1, 3.2, 3.3, 3.4, 3.5, 4.0 or 5.0. In some embodiments, the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3. In some embodiments, the elution buffer has a pH of about 2.7, about 2.75, about 2.8, about 2.85, about 2.9, or about 2.95.

In some embodiments, the elution buffer of step c) has a higher pH than the equilibration buffer. For instance, some antigen binding proteins may not tolerate the low pH of elution buffers having a pH of between, e.g., about 2.0 and about 5.0. In some embodiments, the elution buffer has a pH of about 10.0 or higher. Accordingly, in some embodiments, the elution buffer has a pH of between any of about 10.0 and 12.0, 10.5 and 12.0, 11.0 and 12.0, 10.0 and 11.5, or 10.0 and 11.0. In some embodiments, the elution buffer has a pH of any of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0. In some embodiments, the elution buffer has a pH of between about 10.0 and 12.0.

In some embodiments, the elution buffer comprises acetic acid. In some embodiments, the elution buffer comprises sodium acetate, citrate, or glycine. In some embodiments, the elution buffer comprises acetic acid, sodium acetate, citrate, or glycine at a concentration of between any of about 50 mM and 250 mM, 75 mM and 250 mM, 100 mM and 250 mM, 125 mM and 250 mM, 150 mM and 250 mM, 175 mM and 250 mM, 200 mM and 250 mM, 50 mM and 200 mM, 75 mM and 200 mM, 100 mM and 200 mM, 125 mM and 200 mM, 150 mM and 200 mM, 175 mM and 200 mM, 50 mM and 175 mM, 75 mM and 175 mM, 100 mM and 175 mM, 125 mM and 175 mM, 150 mM and 175 mM, 50 mM and 150 mM, 75 mM and 150 mM, 100 mM and 150 mM, 125 mM and 150 mM, 50 mM and 125 mM, 75 mM and 125 mM, 100 mM and 125 mM, 50 mM and 100 mM, 75 mM and 100 mM, or 50 mM and 75 mM. In some embodiments, the elution buffer comprises acetic acid, sodium acetate, citrate, or glycine at a concentration of any of about 50 mM, 75 mM, 100 mM, 125 mM, 150 mM, 160 mM, 170 mM, 175 mM, 180 mM, 190 mM, 200 mM, 225 mM, or 250 mM.

In some embodiments, the elution buffer comprises about 50 mM acetic acid to about 250 mM acetic acid. In some embodiments, the elution buffer comprises acetic acid at a concentration of between any of about 50 mM and 250 mM, 75 mM and 250 mM, 100 mM and 250 mM, 125 mM and 250 mM, 150 mM and 250 mM, 175 mM and 250 mM, 200 mM and 250 mM, 50 mM and 200 mM, 75 mM and 200 mM, 100 mM and 200 mM, 125 mM and 200 mM, 150 mM and 200 mM, 175 mM and 200 mM, 50 mM and 175 mM, 75 mM and 175 mM, 100 mM and 175 mM, 125 mM and 175 mM, 150 mM and 175 mM, 50 mM and 150 mM, 75 mM and 150 mM, 100 mM and 150 mM, 125 mM and 150 mM, 50 mM and 125 mM, 75 mM and 125 mM, 100 mM and 125 mM, 50 mM and 100 mM, 75 mM and 100 mM, or 50 mM and 75 mM. In some embodiments, the elution buffer comprises acetic acid at a concentration of any of about 50 mM, 75 mM, 100 mM, 125 mM, 150 mM, 160 mM, 170 mM, 175 mM, 180 mM, 190 mM, 200 mM, 225 mM, or 250 mM. In some embodiments, the elution buffer comprises about 150 mM acetic acid. In some embodiments, the elution buffer comprises about 150 mM acetic acid at a pH of about 2.8.

In some embodiments, the antigen binding protein purified by the protein L chromatography is an antibody or immunoadhesin or a fragment thereof. In some embodiments, the VL domain of the antibody or immunoadhesin or a fragment thereof is a λ subtype VL or a κ2 VL. In some embodiments, the VL domain binds poorly to Protein L. In some embodiments, the VL domain is a κ VL and comprises a modification that weakens binding of the VL domain to Protein L. In some embodiments, the modification comprises one or more amino acid substitutions in the VL domain. In some embodiments, the VL domain is a κ2 VL.

In some embodiments, the antigen binding protein binds poorly to Protein L. For instance, in some embodiments, and without being bound by theory, the antigen binding protein employed in the methods described herein bind poorly to Protein L, and the kosmotrope strengthens the binding of the antigen binding protein to Protein L, whereas impurities such as HMWF and LC-dimers may bind more tightly to Protein L than the antigen binding protein, which can result in worse yield and worse quality for the antigen binding protein absent the presence of the kosmotrope. In some embodiments, the antigen binding protein that binds poorly to Protein L comprises a VL domain of the κ2 subtype.

In some embodiments, the antigen binding protein, e.g., antibody or immunoadhesin, binds poorly to Protein L as compared to a reference antigen binding protein. In some embodiments, the reference antigen binding protein is of the same or a similar type of antigen binding protein as the antigen binding protein, e.g., is an antibody. In some embodiments, the antigen binding protein, e.g., antibody or immunoadhesin, binds poorly to Protein L as compared to a reference antigen binding protein that is monoclonal antibody G6-31. In some embodiments, the antigen binding protein, e.g., antibody or immunoadhesin, binds weaker to Protein L than a reference antigen binding protein. In some embodiments, the antigen binding protein, e.g., antibody or immunoadhesin, binds weaker to Protein L than a reference antigen binding protein that is monoclonal antibody G6-31. The G6-31 (also referred to as “G6.31”) monoclonal antibody is an antibody fragment that is an anti-VEGF Fab, which has been widely published on. See, e.g., Korsisaari et al., PNAS, 2007, 104 (25): 10625-10630. In some embodiments, the G6-31 Fab can serve as a reference antigen binding protein for determining whether another antigen binding protein exhibits, e.g., weaker or poorer binding to Protein L, than the G6-31 Fab that is considered to exhibit normal binding to Protein L. In some embodiments, antigen binding proteins that exhibit weak or poor binding to Protein L may be particularly suitable for the methods described herein, which involve the use of a kosmotrope for improving yield and purity of antigen binding proteins of interest that may benefit from strengthening the binding of the antigen binding protein to Protein L.

In some embodiments, the antigen binding protein is a monoclonal antibody. In some embodiments, the antigen binding protein is a human antibody, a chimeric antibody or a humanized antibody. In some embodiments, the antibody is an antibody fragment selected from a Fab, Fab′-SH, Fv, scFv, and (Fab′)2 fragment. In some embodiments, the antibody does not contain an Fc domain.

In some embodiments, the antibody fragment is a Fab. In some embodiments, the Fab comprises a binding site for binding with Protein L that lacks a hydrophobic patch or has a weak hydrophobic patch. In some embodiments, the binding site for binding with Protein L lacks a hydrophobic patch. In some embodiments, the binding site for binding with Protein L has a weak hydrophobic patch. In some embodiments, the binding site for binding with Protein L comprises a hydrophilic patch.

In some embodiments, the antigen binding protein that binds poorly to Protein L comprises a binding site for binding with Protein L that lacks a hydrophobic patch or has a weak hydrophobic patch. In some embodiments, the binding site for binding with Protein L has a weak hydrophobic patch as compared to the binding site for binding with Protein L of a reference antigen binding protein that is monoclonal antibody G6-31.

In some embodiments, the antigen binding protein that binds poorly to Protein L comprises a hydrophilic patch.

In some embodiments, the binding affinity (EC50) and/or the dissociation constant of the antigen binding protein, e.g., antibody or immunoadhesin, to Protein L has a value that is higher than the value of the binding affinity (EC50) and/or the dissociation constant of a reference antigen binding protein that is monoclonal antibody G6-31 to Protein L. In some embodiments, the binding affinity (EC50) and/or the dissociation constant of the antigen binding protein, e.g., antibody or immunoadhesin, to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.

In some embodiments, the antigen binding protein, e.g., Fab, comprises a VL domain, and the binding site is contained within the VL domain.

In some embodiments, the antibody is a multispecific antibody. In some embodiments, the multispecific antibody is a bispecific antibody. In some embodiments, the multispecific antibody comprises the VL domain and a second VL domain, and wherein the VL domain is a kappa (κ) VL. In some embodiments, the VL domain is a κ2 VL. In some embodiments, the VL domain binds poorly to Protein L. In some embodiments, the VL domain binds poorly to Protein L and the second VL domain does not bind to Protein L. In some embodiments, the antigen binding protein, e.g., antibody or immunoadhesin, comprises a VL domain and binds poorly to Protein L as compared to a reference antigen binding protein comprising a VL domain. In some embodiments, the reference antigen binding protein is of the same or a similar type of antigen binding protein as the antigen binding protein, e.g., is an antibody. In some embodiments, the antigen binding protein, e.g., antibody or immunoadhesin, comprises a VL domain and binds poorly to Protein L as compared to a reference antigen binding protein comprising a VL domain that is monoclonal antibody G6-31. In some embodiments, the antigen binding protein, e.g., antibody or immunoadhesin, comprises a VL domain and binds weaker to Protein L than a reference antigen binding protein comprising a VL domain. In some embodiments, the antigen binding protein, e.g., antibody or immunoadhesin, comprises a VL domain and binds weaker to Protein L than a reference antigen binding protein comprising a VL domain that is monoclonal antibody G6-31. In some embodiments, the VL domain binds poorly to Protein L as compared to the binding of the VL domain of a reference antigen binding protein that is monoclonal antibody G6-31 to Protein L.

In some embodiments, the second VL domain is a lambda (λ) VL. In some embodiments, the VL domain is a κ VL and comprises a modification that weakens binding of the VL domain to Protein L. In some embodiments, the modification comprises one or more amino acid substitutions in the VL domain. In some embodiments, the VL domain is a κ2 VL.

In some embodiments, the VL domain is a λ subtype VL or a κ2 VL.

### Protein L Chromatography

Protein L is a cell wall protein of the bacterium Peptostreptococcus magnus (Björck et al. (1988) J. Immunol. 140:1194-1197) that binds to the variable region of the kappa light chain without interfering with the antigen binding site (Nilson et al. (1992) J Biol Chem. 267:2234-2239) of an antibody or antibody fragment. Protein L interacts with FW1 in V-region of a kappa light chain, and its binding is restricted to VL of κ1, κ3 and κ4 subtypes but does not bind or binds weakly to VL of the κ2 subtype. Commercially available Protein L chromatography materials include, but are not limited to a Pierce™ Protein L chromatography cartridge, a Capto™ L chromatography, HiTrap® Protein L chromatography, a KanCap™ ML chromatography, a TOYOPEARL® AF-rProtein L-650F chromatography, or a MabSelect™ V_{L} chromatography.

### Additional Steps

In some aspects, the present invention provides additional steps involved or associated with purification of an antigen binding protein as described herein. Additional steps involved or associated with the purification, and methods for conducting such steps, are known. See, e.g., Liu et al., mAbs, 2, 2010, which is hereby incorporated by reference in its entirety.

In some embodiments, the purification of the antigen binding protein further comprises a sample processing step, such as a sample preparation step. In some embodiments, the purification of the antigen binding protein further comprises a clarification step, such as to clarify HCCF. In some embodiments, the purification of the antigen binding protein further comprises a host cell and host cell debris removal step, such as to remove host cells and host cell debris from a sample and/or a composition obtained from the purification platform. In some embodiments, the purification of the antigen binding protein further comprises a centrifugation step. In some embodiments, the purification of the antigen binding protein further comprises a sterile filtration step. In some embodiments, the purification of the antigen binding protein further comprises a tangential flow micro-filtration step. In some embodiments, the purification of the antigen binding protein further comprises a flocculation/precipitation step.

### Antigen Binding Proteins

In some aspects, the methods of protein L chromatography using a wash buffer comprising a kosmotrope is useful for purifying and concentrating an antigen binding protein comprising a VL domain from an antigen binding protein preparation. In some embodiments, the antigen binding protein preparation is derived from a host cell preparation. In some embodiments, the host cell preparation is a host cell culture fluid (HCCF). In some embodiments, the antigen binding protein preparation comprises a portion of a host cell culture fluid. In some embodiments, the antigen binding protein preparation is derived from a host cell culture fluid. In some embodiments, the antigen binding protein preparation comprises a host cell. In some embodiments, the antigen binding protein preparation comprises a component of a host cell, such as host cell debris. In some embodiments, the host cell is a bacterial cell. In some embodiments, the host cell is an insect cell. In some embodiments, the host cell is a mammalian cell. In some embodiments, the host cell is a Chinese hamster ovary (CHO) cell. In some embodiments, the host cell is an E. coli cell.

### Antigen Binding Proteins

The antigen binding proteins to be purified by protein L chromatography including the use of a kosmotrope in a wash buffer using the methods described herein are generally produced using recombinant techniques. Methods for producing recombinant proteins are described, e.g., in U.S. Pat. Nos. 5,534,615 and 4,816,567, specifically incorporated herein by reference. In some embodiments, the protein of interest is produced in a CHO cell (see, e.g. WO 94/11026). In some embodiments, the polypeptide of interest is produced in an E. coli cell. See, e.g., U.S. Pat. Nos. 5,648,237; 5,789,199, and 5,840,523, which describes translation initiation region (TIR) and signal sequences for optimizing expression and secretion. See also Charlton, Methods in Molecular Biology, Vol. 248 (B. K. C. Lo, ed., Humana Press, Totowa, N.J., 2003), pp. 245-254, describing expression of polypeptide fragments in E. coli. When using recombinant techniques, the polypeptides can be produced intracellularly, in the periplasmic space, or directly secreted into the medium.

The antigen binding proteins may be recovered from culture medium or from host cell lysates. Cells employed in expression of the antigen binding proteins can be disrupted by various physical or chemical means, such as freeze-thaw cycling, sonication, mechanical disruption, or cell lysing agents. If the antigen binding proteins is produced intracellularly, as a first step, the particulate debris, either host cells or lysed fragments, are removed, for example, by centrifugation or ultrafiltration. Carter et al., Bio/Technology 10:163-167 (1992) describe a procedure for isolating polypeptides which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antigen binding proteins is secreted into the medium, supernatants from such expression systems are generally first concentrated using a commercially available polypeptide concentration filter, for example, an Amicon® or Millipore Pellicon® ultrafiltration unit. A protease inhibitor such as PMSF may be included in any of the foregoing steps to inhibit proteolysis and antibiotics may be included to prevent the growth of adventitious contaminants.

In some embodiments, the antigen binding proteins have been purified or partially purified prior to analysis by the methods of the invention. For example, the antigen binding proteins of the methods is in an eluent from an affinity chromatography, a cation exchange chromatography, an anion exchange chromatography, a mixed mode chromatography and a hydrophobic interaction chromatography.

Examples of antigen binding proteins to be purified by protein L chromatography including the use of a kosmotrope in a wash buffer using the methods described herein include but are not limited to immunoglobulins, immunoadhesins, antibodies, and immunoconjugates.

### (A) Antibodies

In some embodiments of any of the methods described herein, the antigen binding protein is an antibody or immunoadhesin.

### (i) Monoclonal Antibodies

In some embodiments, the antibodies are monoclonal antibodies. Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature 256:495 (1975), or may be made by recombinant DNA methods (U.S. Pat. No. 4,816,567).

In the hybridoma method, a mouse or other appropriate host animal, such as a hamster, is immunized as herein described to elicit lymphocytes that produce or are capable of producing antibodies that will specifically bind to the polypeptide used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In some embodiments, the myeloma cells are those that fuse efficiently, support stable high-level production of antibody by the selected antibody-producing cells, and are sensitive to a medium such as HAT medium. Among these, in some embodiments, the myeloma cell lines are murine myeloma lines, such as those derived from MOPC-21 and MPC-11 mouse tumors available from the Salk Institute Cell Distribution Center, San Diego, California USA, and SP-2 or X63-Ag8-653 cells available from the American Type Culture Collection, Rockville, Maryland USA. Human myeloma and mouse-human heteromyeloma cell lines also have been described for the production of human monoclonal antibodies (Kozbor, J. Immunol. 133:3001 (1984); Brodeur et al., Monoclonal Antibody Production Techniques and Applications pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem. 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, polypeptide A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). In some embodiments, the hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin polypeptide, to obtain the synthesis of monoclonal antibodies in the recombinant host cells. Review articles on recombinant expression in bacteria of DNA encoding the antibody include Skerra et al., Curr. Opinion in Immunol. 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature 348:552-554 (1990). Clackson et al., Nature 352:624-628 (1991) and Marks et al., J. Mol. Biol. 222:581-597 (1991) describe the isolation of murine and human antibodies, respectively, using phage libraries. Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison et al., Proc. Natl Acad. Sci. USA 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In some embodiments of any of the methods described herein, the antibody is IgA, IgD, IgE, IgG, or IgM. In some embodiments, the antibody is an IgG monoclonal antibody.

### (ii) Humanized Antibodies

In some embodiments, the antibody is a humanized antibody. Methods for humanizing non-human antibodies have been described in the art. In some embodiments, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol. 151:2296 (1993); Chothia et al., J. Mol. Biol. 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA 89:4285 (1992); Presta et al., J. Immunol. 151:2623 (1993)).

It is further important that antibodies be humanized with retention of high affinity for the antigen and other favorable biological properties. To achieve this goal, in some embodiments of the methods, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iii) Human Antibodies

In some embodiments, the antibody is a human antibody. As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immuno. 7:33 (1993); and U.S. Pat. Nos. 5,591,669; 5,589,369; and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat polypeptide gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

Human antibodies may also be generated by in vitro activated B cells (see U.S. Pat. Nos. 5,567,610 and 5,229,275).

### (iv) Antibody Fragments

In some embodiments, the antibody is an antibody fragment. In some embodiments, the antibody fragment is a Fab, Fab′-SH, Fv, scFv, or (Fab′)2 fragment. In some embodiments, the antibody fragment is a Fab. In some embodiments, the antibody is an antibody fragment comprising an Fc receptor binding domain. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science 229:81 (1985)). However, these fragments can now be produced directly by recombinant host cells. For example, the antibody fragments can be isolated from the antibody phage libraries discussed above.

In some embodiments, fragments of the antibodies described herein are provided. In some embodiments, the antibody fragment is an antigen binding fragment. In some embodiments, the antibody fragment is an antigen binding fragment comprising an Fc receptor binding domain. In some embodiments, the antibody fragment is an antigen binding fragment comprising an Fcγ receptor binding domain.

### (v) Bispecific Antibodies

In some embodiments, the antibody is a bispecific antibody. Bispecific antibodies are antibodies that have binding specificities for at least two different epitopes. Exemplary bispecific antibodies may bind to two different epitopes. Alternatively, a bispecific antibody binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the cell. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. In some embodiments, the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers that are recovered from recombinant cell culture. In some embodiments, the interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 0308936). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148 (5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152:5368 (1994).

Antibodies with more than two valencies are contemplated. For example, trispecific antibodies can be prepared. Tutt et al., J. Immunol. 147:60 (1991).

### (v) Multivalent Antibodies

In some embodiments, the antibodies are multivalent antibodies. A multivalent antibody may be internalized (and/or catabolized) faster than a bivalent antibody by a cell expressing an antigen to which the antibodies bind. The antibodies provided herein can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1) n-VD2-(X2) n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In some embodiments, the antibody is a multispecific antibody. Example of multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. In some embodiments, the multispecific antibody comprises a V_{H} domain and a second V_{H} domain, which each bind to a different epitope. In some embodiment that antibody has polyepitopic specificity; for example, the ability to specifically bind to two or more different epitopes on the same or different target(s). In some embodiments, the antibodies are monospecific; for example, an antibody that binds only one epitope. According to one embodiment the multispecific antibody is an IgG antibody that binds to each epitope with an affinity of 5 μM to 0.001 μM, 3 μM to 0.001 μM, 1 μM to 0.001 μM, 0.5 μM to 0.001 μM, or 0.1 μM to 0.001 μM.

### (vi) Other Antibody Modifications

It may be desirable to modify the antibody provided herein with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J., Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement mediated lysis and ADCC capabilities. See Stevenson et al., Anti-Cancer Drug Design 3:219-230 (1989).

For increasing serum half-life of the antibody, amino acid alterations can be made in the antibody as described in US 2006/0067930, which is hereby incorporated by reference in its entirety.

### (B) Polypeptide Variants and Modifications

Amino acid sequence modification(s) of the polypeptides (e.g., antigen binding proteins), including antibodies, described herein may be used in the methods of purifying polypeptides (e.g., antigen binding proteins) described herein.

### (i) Variant Polypeptides

“Polypeptide variant” means a polypeptide (e.g., an antigen binding protein), preferably an active polypeptide, as defined herein having at least about 80% amino acid sequence identity with a full-length native sequence of the polypeptide, a polypeptide sequence lacking the signal peptide, an extracellular domain of a polypeptide, with or without the signal peptide. Such polypeptide variants include, for instance, polypeptides wherein one or more amino acid residues are added, or deleted, at the N or C-terminus of the full-length native amino acid sequence. Ordinarily, a TAT polypeptide variant will have at least about 80% amino acid sequence identity, alternatively at least about any of 85%, 90%, 95%, 96%, 97%, 98%, or 99% amino acid sequence identity, to a full-length native sequence polypeptide sequence, a polypeptide sequence lacking the signal peptide, an extracellular domain of a polypeptide, with or without the signal peptide. Optionally, variant polypeptides will have no more than one conservative amino acid substitution as compared to the native polypeptide sequence, alternatively no more than about any of 2, 3, 4, 5, 6, 7, 8, 9, or 10 conservative amino acid substitutions as compared to the native polypeptide sequence.

The variant polypeptide may be truncated at the N-terminus or C-terminus, or may lack internal residues, for example, when compared with a full length native polypeptide. Certain variant polypeptides may lack amino acid residues that are not essential for a desired biological activity. These variant polypeptides with truncations, deletions, and insertions may be prepared by any of a number of conventional techniques. Desired variant polypeptides may be chemically synthesized. Another suitable technique involves isolating and amplifying a nucleic acid fragment encoding a desired variant polypeptide, by polymerase chain reaction (PCR). Oligonucleotides that define the desired termini of the nucleic acid fragment are employed at the 5′ and 3′ primers in the PCR. Preferably, variant polypeptides share at least one biological and/or immunological activity with the native polypeptide disclosed herein.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

For example, it may be desirable to improve the binding affinity and/or other biological properties of the polypeptide (e.g., antigen binding protein). Amino acid sequence variants of the polypeptide are prepared by introducing appropriate nucleotide changes into the antibody nucleic acid, or by peptide synthesis. Such modifications include, for example, deletions from, and/or insertions into and/or substitutions of, residues within the amino acid sequences of the polypeptide. Any combination of deletion, insertion, and substitution is made to arrive at the final construct, provided that the final construct possesses the desired characteristics. The amino acid changes also may alter post-translational processes of the polypeptide (e.g., antibody), such as changing the number or position of glycosylation sites.

Guidance in determining which amino acid residue may be inserted, substituted or deleted without adversely affecting the desired activity may be found by comparing the sequence of the polypeptide with that of homologous known polypeptide molecules and minimizing the number of amino acid sequence changes made in regions of high homology.

A useful method for identification of certain residues or regions of the polypeptide (e.g., antigen binding protein) that are preferred locations for mutagenesis is called “alanine scanning mutagenesis” as described by Cunningham and Wells, Science 244:1081-1085 (1989). Here, a residue or group of target residues are identified (e.g., charged residues such as Arg, Asp, His, Lys, and Glu) and replaced by a neutral or negatively charged amino acid (most preferably Alanine or Polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table 1 below under the heading of “exemplary substitutions.” If such substitutions result in a change in biological activity, then more substantial changes, denominated “substitutions” in the Table 1, or as further described below in reference to amino acid classes, may be introduced and the products screened.

TABLE 1

| Original | | Exemplary |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, Biochemistry second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser(S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the polypeptide to improve its stability (particularly where the antibody is an antibody fragment such as an Fv fragment).

A particularly preferred type of substitutional variant involves substituting one or more hypervariable region residues of a parent antibody (e.g., a humanized antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and target. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the polypeptide (e.g., antigen binding protein) alters the original glycosylation pattern of the antibody. The polypeptide may comprise non-amino acid moieties. For example, the polypeptide may be glycosylated. Such glycosylation may occur naturally during expression of the polypeptide in the host cell or host organism, or may be a deliberate modification arising from human intervention. By altering is meant deleting one or more carbohydrate moieties found in the polypeptide, and/or adding one or more glycosylation sites that are not present in the polypeptide.

Glycosylation of polypeptide is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Removal of carbohydrate moieties present on the polypeptide may be accomplished chemically or enzymatically or by mutational substitution of codons encoding for amino acid residues that serve as targets for glycosylation. Enzymatic cleavage of carbohydrate moieties on polypeptides can be achieved by the use of a variety of endo- and exo-glycosidases.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains, acetylation of the N-terminal amine, and amidation of any C-terminal carboxyl group.

### (ii) Chimeric Polypeptides

The polypeptide (e.g., antigen binding protein) described herein may be modified in a way to form chimeric molecules comprising the polypeptide fused to another, heterologous polypeptide or amino acid sequence. In some embodiments, a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag.

In an alternative embodiment, the chimeric molecule may comprise a fusion of the polypeptide with an immunoglobulin or a particular region of an immunoglobulin. A bivalent form of the chimeric molecule is referred to as an “immunoadhesin.”

As used herein, the term “immunoadhesin” designates antibody-like molecules which combine the binding specificity of a heterologous polypeptide with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2, IgG3, or IgG4 subtypes, IgA (including IgAQ1 and IgA2), IgE, IgD or IgM.

The Ig fusions preferably include the substitution of a soluble (transmembrane domain deleted or inactivated) form of a polypeptide in place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule.

(iii) Polypeptide Conjugates

The antigen binding protein for use in the methods described herein may be conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such conjugates can be used. In addition, enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated polypeptides. Examples include ²¹²Bi, ¹³¹I, ¹³¹In, ⁹⁰Y, and ¹⁸⁶Re. Conjugates of the polypeptide and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the polypeptide.

Conjugates of an antigen binding protein and one or more small molecule toxins, such as a calicheamicin, maytansinoids, a trichothene, and CC1065, and the derivatives of these toxins that have toxin activity, are also contemplated herein.

Maytansinoids are mitototic inhibitors which act by inhibiting tubulin polymerization. Maytansine was first isolated from the east African shrub Maytenus serrata. Subsequently, it was discovered that certain microbes also produce maytansinoids, such as maytansinol and C-3 maytansinol esters. Synthetic maytansinol and derivatives and analogues thereof are also contemplated. There are many linking groups known in the art for making polypeptide-maytansinoid conjugates, including, for example, those disclosed in U.S. Pat. No. 5,208,020. The linking groups include disufide groups, thioether groups, acid labile groups, photolabile groups, peptidase labile groups, or esterase labile groups, as disclosed in the above-identified patents, disulfide and thioether groups being preferred.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Another conjugate of interest comprises an antigen binding protein conjugated to one or more calicheamicin molecules. The calicheamicin family of antibiotics is capable of producing double-stranded DNA breaks at sub-picomolar concentrations. For the preparation of conjugates of the calicheamicin family, see, e.g., U.S. Pat. No. 5,712,374. Structural analogues of calicheamicin which may be used include, but are not limited to, γ₁¹, α₂¹, α₃¹, N-acetyl-γ₁¹, PSAG and θ₁¹. Another anti-tumor drug that the antibody can be conjugated is QFA which is an antifolate. Both calicheamicin and QFA have intracellular sites of action and do not readily cross the plasma membrane. Therefore, cellular uptake of these agents through polypeptide (e.g., antibody) mediated internalization greatly enhances their cytotoxic effects.

Other antitumor agents that can be conjugated to the polypeptides described herein include BCNU, streptozoicin, vincristine and 5-fluorouracil, the family of agents known collectively LL-E33288 complex, as well as esperamicins.

In some embodiments, the antigen binding protein may be a conjugate between a polypeptide and a compound with nucleolytic activity (e.g., a ribonuclease or a DNA endonuclease such as a deoxyribonuclease; DNase).

In yet another embodiment, the antigen binding protein may be conjugated to a “receptor” (such streptavidin) for utilization in tumor pre-targeting wherein the polypeptide receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

In some embodiments, the antigen binding protein may be conjugated to a prodrug-activating enzyme which converts a prodrug (e.g., a peptidyl chemotherapeutic agent) to an active anti-cancer drug. The enzyme component of the immunoconjugate includes any enzyme capable of acting on a prodrug in such a way so as to convert it into its more active, cytotoxic form.

Enzymes that are useful include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs into free active drugs.

### (iv) Other

Another type of covalent modification of the antigen binding protein comprises linking the antigen binding protein to one of a variety of nonproteinaceous polymers, e.g., polyethylene glycol, polypropylene glycol, polyoxyalkylenes, or copolymers of polyethylene glycol and polypropylene glycol. The antigen binding protein also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 18th edition, Gennaro, A. R., Ed., (1990).

The antigen binding protein for use in the methods described herein may comprise a modification that weakens binding of the VL domain to Protein L. In some embodiments, the modification comprises one or more amino acid substitutions in the VL domain. In some embodiments, the VL domain is a κ2 VL. In some embodiments, the modification comprises a S12P substitution in the VL domain.

### Obtaining Polypeptides for Use in the Methods

The antigen binding proteins used in the methods of purification described herein may be obtained using methods well-known in the art, including the recombination methods. The following sections provide guidance regarding these methods.

### (A) Polynucleotides

“Polynucleotide,” or “nucleic acid,” as used interchangeably herein, refer to polymers of nucleotides of any length, and include DNA and RNA.

Polynucleotides encoding polypeptides may be obtained from any source including, but not limited to, a cDNA library prepared from tissue believed to possess the polypeptide mRNA and to express it at a detectable level. Accordingly, polynucleotides encoding polypeptide can be conveniently obtained from a cDNA library prepared from human tissue. The polypeptide-encoding gene may also be obtained from a genomic library or by known synthetic procedures (e.g., automated nucleic acid synthesis).

For example, the polynucleotide may encode an entire immunoglobulin molecule chain, such as a light chain or a heavy chain. A complete heavy chain includes not only a heavy chain variable region (V_{H}) but also a heavy chain constant region (C_{H}), which typically will comprise three constant domains: C_{H}1, C_{H}2 and C_{H}3; and a “hinge” region. In some situations, the presence of a constant region is desirable.

Other antigen binding proteins which may be encoded by the polynucleotide include antigen-binding antibody fragments such as single domain antibodies (“dAbs”), Fv, scFv, Fab′ and F(ab′)₂ and “minibodies.” Minibodies are (typically) bivalent antibody fragments from which the C_{H}1 and C_{K} or C_{L} domain has been excised. As minibodies are smaller than conventional antibodies they should achieve better tissue penetration in clinical/diagnostic use, but being bivalent they should retain higher binding affinity than monovalent antibody fragments, such as dAbs. Accordingly, unless the context dictates otherwise, the term “antibody” as used herein encompasses not only whole antibody molecules but also antigen-binding antibody fragments of the type discussed above. Preferably each framework region present in the encoded polypeptide will comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions.

### Pharmaceutical Compositions

In some aspects, the present disclosure provides pharmaceutical compositions comprising an antigen binding protein obtained from the purification processes described herein (e.g., protein L chromatography using a kosmotrope as described herein). In some embodiments, the pharmaceutical composition is a purified composition. In some embodiments, the pharmaceutical composition is a sterile pharmaceutical composition.

The pharmaceutical composition may be prepared for storage by mixing an antigen binding protein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Kits

In some aspects of the invention, a kit for use in the methods to purify antigen binding proteins as described herein. In some embodiments, the kit includes one or more of a protein L chromatography material, an equilibration buffer, a wash buffer, an elution buffer and a kosmotrope. In some embodiments, the kit further provides instructions for its use. The containers hold the formulations and the labels on, or associated with, the containers may indicate directions for use. The article of manufacture may further include other materials desirable from a commercial and user standpoint, including other buffers, diluents, cultureware, reagents for detecting reporter molecules, and package inserts with instructions for use. The kit may further include other materials desirable from a commercial and user standpoint, including other buffers, diluents, cultureware, reagents for detecting reporter molecules, and package inserts with instructions for use.

### Exemplary Embodiments

Provided herein are the following exemplary embodiments:
1. A method for purifying an antigen binding protein comprising a VL domain, the method comprising:
   a) binding the antigen binding protein to a Protein L chromatography material,
   b) washing the Protein L chromatography material with a wash buffer, wherein the wash buffer comprises an equilibration buffer and a kosmotrope, and
   c) eluting the antigen binding protein from the Protein L chromatography material with an elution buffer.
2. A method for improving Protein L purification of an antigen binding protein comprising a VL domain, the method comprising:
   a) binding the antigen binding protein to a Protein L chromatography material,
   b) washing the Protein L chromatography material with a wash buffer, wherein the wash buffer comprises an equilibration buffer and a kosmotrope, and
   c) eluting the antigen binding protein from the Protein L chromatography material with an elution buffer.
3. The method of embodiment 2, wherein the yield and/or purity of the antigen binding protein is increased compared to Protein L chromatography where the wash buffer does not comprise a kosmotrope.
4. The method of any one of embodiments 1-3, wherein the equilibration buffer comprises Tris, MES, MOPS, or EDTA.
5. The method of any one of embodiments 1-4, wherein the equilibration buffer comprises Tris and NaCl.
6. The method of embodiment 4 or embodiment 5, wherein the Tris is present in the equilibration buffer at a concentration of about 10 mM to about 50 mM, or about 25 mM.
7. The method of embodiment 5 or embodiment 6, wherein the NaCl is present in the equilibration buffer at a concentration of about 10 mM to about 50 mM, or about 25 mM.
8. The method of any one of embodiments 1-7, wherein the equilibration buffer has a pH of about 4 to about 10.
9. The method of any one of embodiments 1-8, wherein the equilibration buffer has a pH of about 6.5 to about 8.5, or about 7.7.
10. The method of any one of embodiments 1-9, wherein the equilibration buffer has a pH of about 7 to about 8.
11. The method of any one of embodiments 1-10, wherein the wash buffer has a pH of about 4 to about 10.
12. The method of any one of embodiments 1-11, wherein the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5.
13. The method of any one of embodiments 1-12, wherein the wash buffer has a pH of about 4, about 4.5, about 5, about 5.5, about 6, about 6.5, about 7, about 7.5, about 8, about 8.5, about 9, about 9.5, or about 10.
14. The method of any one of embodiments 1-12, wherein the wash buffer has a pH of about 7.0, about 7.1, about 7.2, about 7.3, about 7.4, about 7.5, about 7.6, about 7.7, about 7.8, about 7.9, or about 8.0.
15. The method of any one of embodiments 1-14, wherein the antigen binding protein is loaded onto the protein L chromatography material in step a) as a host cell culture supernatant.
16. The method of any one of embodiments 1-14, wherein the antigen binding protein is loaded onto the protein L chromatography material in step a) as a purified polypeptide solution.
17. The method of any one of embodiments 1-14, wherein the antigen binding protein is loaded onto the protein L chromatography material in step a) as a mixture from a prior purification step.
18. The method of any one of embodiments 1-14, wherein the antigen binding protein is formulated in equilibration buffer prior to loading onto the protein L chromatography material in step a).
19. The method of any one of embodiments 1-14 and 18, wherein the antigen binding protein is formulated in equilibration buffer and the kosmotrope prior to loading onto the protein L chromatography material in step a).
20. The method of any one of embodiments 1-19, wherein the kosmotrope is a phosphate salt or a sulfate salt.
21. The method of any one of embodiments 1-20, wherein the kosmotrope is potassium phosphate, sodium sulfate, or ammonium sulfate.
22. The method of any one of embodiments 1-21, wherein the concentration of the kosmotrope in the equilibration buffer or the wash buffer is about 100 mM to about 800 mM.
23. The method of any one of embodiments 1-21, wherein the concentration of the kosmotrope in the equilibration buffer and the wash buffer is about 100 mM to about 800 mM.
24. The method of any one of embodiments 1-23, wherein the concentration of the kosmotrope in the equilibration buffer or the wash buffer is about 120 mM to about 600 mM.
25. The method of any one of embodiments 1-24, wherein the concentration of the kosmotrope in the equilibration buffer or the wash buffer is about 120 mM, about 240 mM, about 360 mM, about 480 mM, or about 600 mM.
26. The method of any one of embodiments 1-21, wherein the concentration of the kosmotrope in the equilibration buffer and/or the wash buffer is about 100 mM to about 800 mM, about 100 mM to about 700 mM, about 100 mM to about 600 mM, about 100 mM to about 500 mM, about 200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM.
27. The method of any one of embodiments 1-21, wherein the concentration of the kosmotrope in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM.
28. The method of any one of embodiments 1-27, wherein the kosmotrope is sodium sulfate.
29. The method of any one of embodiments 1-27, wherein the kosmotrope is potassium phosphate.
30. The method of any one of embodiments 1-27, wherein the kosmotrope is ammonium sulfate.
31. The method of any one of embodiments 1-30, wherein the elution buffer has a lower pH than the equilibration buffer.
32. The method of any one of embodiments 1-31, wherein the elution buffer has a pH of about 2.0 to about 4.0, or about 2.5 to about 3.0.
33. The method of any one of embodiments 1-32, wherein the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3.
34. The method of any one of embodiments 1-32, wherein the elution buffer has a pH of about 2.7, about 2.75, about 2.8, about 2.85, about 2.9, or about 2.95.
35. The method of any one of embodiments 1-30, wherein the elution buffer has a higher pH than the equilibration buffer.
36. The method of embodiment 35, wherein the elution buffer has a pH of about 10.0 to about 12.0.
37. The method of any one of embodiments 1-36, wherein the elution buffer comprises acetic acid.
38. The method of embodiment 37, wherein the elution buffer comprises about 50 mM acetic acid to about 250 mM acetic acid.
39. The method of embodiment 37 or embodiment 38, wherein the elution buffer comprises about 150 mM acetic acid at a pH of about 2.8.
40. The method of any one of embodiments 1-36, wherein the elution buffer comprises sodium acetate, citrate, or glycine.
41. The method of any one of embodiments 1-40, wherein the antigen binding protein binds poorly to Protein L.
42. The method of any one of embodiments 1-41, wherein the antigen binding protein binds poorly to Protein L as compared to a reference antigen binding protein that is monoclonal antibody G6-31.
43. The method of any one of embodiments 1-41, wherein the antigen binding protein binds weaker to Protein L than a reference antigen binding protein that is monoclonal antibody G6-31.
44. The method of any one of embodiments 1-43, wherein the antigen binding protein is an antibody or immunoadhesin or a fragment thereof.
45. The method of any one of embodiments 1-44, wherein the antigen binding protein is a monoclonal antibody.
46. The method of any one of embodiments 1-45, wherein the antigen binding protein is a human antibody, a chimeric antibody or a humanized antibody.
47. The method of any one of embodiments 44-46, wherein the antibody is an antibody fragment selected from a Fab, Fab′-SH, Fv, scFv, and (Fab′)2 fragment.
48. The method of embodiment 47, wherein the antibody fragment is a Fab.
49. The method of embodiment 48, wherein the Fab comprises a binding site for binding with Protein L that lacks a hydrophobic patch or has a weak hydrophobic patch.
50. The method of embodiment 49, wherein the binding site for binding with Protein L lacks a hydrophobic patch.
51. The method of embodiment 49, wherein the binding site for binding with Protein L has a weak hydrophobic patch.
52. The method of any one of embodiments 49-51, wherein the binding site for binding with Protein L has a weak hydrophobic patch as compared to the binding site for binding with Protein L of a reference antigen binding protein that is monoclonal antibody G6-31.
53. The method of any one of embodiments 48-52, wherein the binding site for binding with Protein L comprises a hydrophilic patch.
54. The method of any one of embodiments 1-53, wherein the binding affinity (EC50) and/or the dissociation constant of the antigen binding protein to Protein L has a value that is higher than the value of the binding affinity (EC50) and/or the dissociation constant of a reference antigen binding protein that is monoclonal antibody G6-31 to Protein L.
55. The method of embodiment 54, wherein the binding affinity (EC50) and/or the dissociation constant of the antigen binding protein to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.
56. The method of any one of embodiments 44-55, wherein the antibody does not contain an Fc domain.
57. The method of any one of embodiments 44-46, wherein the antibody is a multispecific antibody.
58. The method of embodiment 57, wherein the multispecific antibody comprises the VL domain and a second VL domain, and wherein the VL domain is a kappa (κ) VL.
59. The method of embodiment 58, wherein the VL domain is a κ2 VL.
60. The method of any one of embodiments 57-59, wherein the VL domain binds poorly to Protein L.
61. The method of any one of embodiments 58-60, wherein the VL domain binds poorly to Protein L and the second VL domain does not bind to Protein L.
62. The method of any one of embodiments 58-61, wherein the second VL domain is a lambda (λ) VL.
63. The method of any one of embodiments 1-56, wherein the VL domain is a λ subtype VL or a κ VL.
64. The method of embodiment 63, wherein the VL domain binds poorly to Protein L.
65. The method of any one of embodiments 60-64, wherein the VL domain binds poorly to Protein L as compared to the binding of the VL domain of a reference antigen binding protein that is monoclonal antibody G6-31 to Protein L.
66. The method of any one of embodiments 63-65, wherein the VL domain is a κ VL and comprises a modification that weakens binding of the VL domain to Protein L.
67. The method of embodiment 66, wherein the modification comprises one or more amino acid substitutions in the VL domain.
68. The method of any one of embodiments 63-67, wherein the VL domain is a κ2 VL.
69. The method of any one of embodiments 1-48 and 56-68, wherein the antigen binding protein comprises a binding site for binding with Protein L that lacks a hydrophobic patch or has a weak hydrophobic patch.
70. The method of embodiment 69, wherein the binding site for binding with Protein L lacks a hydrophobic patch.
71. The method of embodiment 69, wherein the binding site for binding with Protein L has a weak hydrophobic patch.
72. The method of any one of embodiments 69-71, wherein the binding site for binding with Protein L has a weak hydrophobic patch as compared to the binding site for binding with Protein L of a reference antigen binding protein that is monoclonal antibody G6-31.
73. The method of any one of embodiments 69-71, wherein the binding site for binding with Protein L comprises a hydrophilic patch.
74. The method of any one of embodiments 1-73, wherein the protein L chromatography material is a Pierce™ Protein L chromatography cartridge, a Capto™ L chromatography, HiTrap® Protein L chromatography, a KanCap™ L chromatography, a TOYOPEARL® AF-rProtein L-650F chromatography, or a MabSelect™ V_{L} chromatography.
75. A composition comprising an antigen binding protein purified by a method comprising the method of any one of embodiments 1-74.
76. The composition of embodiment 75, wherein the composition comprises one or more pharmaceutical excipients.
77. A kit for purifying an antigen binding protein comprising a V_{L} domain, wherein the kit comprises a Protein L chromatography material and a kosmotrope.
78. The kit of embodiment 77, wherein the kit further comprises an equilibration buffer.
79. The kit of embodiment 78, wherein the equilibration buffer comprises Tris, MES, MOPS, or EDTA.
80. The kit of embodiment 78 or embodiment 79, wherein the equilibration buffer comprises Tris and NaCl.
81. The kit of embodiment 79 or embodiment 80, wherein the Tris is present in the equilibration buffer at a concentration of about 10 mM to about 50 mM or about 25 mM.
82. The kit of embodiment 80 or embodiment 81, wherein the NaCl is present in the equilibration buffer at a concentration of about 10 mM to about 50 mM or about 25 mM.
83. The kit of any one of embodiments 78-82, wherein the equilibration buffer has a pH of about 4 to about 10.
84. The kit of any one of embodiments 78-83, wherein the equilibration buffer has a pH of about 6.5 to about 8.5, or about 7.7.
85. The kit of any one of embodiments 78-84, wherein the equilibration buffer has a pH of about 7 to about 8.
86. The kit of any one of embodiments 77-85, wherein the kit further comprises a wash buffer.
87. The kit of embodiment 86, wherein the wash buffer has a pH of about 4 to about 10.
88. The kit of embodiment 86 or embodiment 87, wherein the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5.
89. The kit of any one of embodiments 86-88, wherein the wash buffer has a pH of about 4, about 4.5, about 5, about 5.5, about 6, about 6.5, about 7, about 7.5, about 8, about 8.5, about 9, about 9.5, or about 10.
90. The kit of any one of embodiments 86-88, wherein the wash buffer has a pH of about 7.0, about 7.1, about 7.2, about 7.3, about 7.4, about 7.5, about 7.6, about 7.7, about 7.8, about 7.9, or about 8.0.
91. The kit of any one of embodiments 86-90, wherein the wash buffer comprises the equilibration buffer and the kosmotrope.
92. The kit of any one of embodiments 78-91, wherein the equilibration buffer further comprises the kosmotrope.
93. The kit of any one of embodiments 77-92, wherein the kosmotrope is a phosphate salt or a sulfate salt.
94. The kit of any one of embodiments 77-93, wherein the kosmotrope is potassium phosphate, sodium sulfate, or ammonium sulfate.
95. The kit of any one embodiments 78-94, wherein the concentration of the kosmotrope in the equilibration buffer or the wash buffer is about 100 mM to about 800 mM.
96. The kit of any one of embodiments 78-94, wherein the concentration of the kosmotrope in the equilibration buffer and the wash buffer is about 100 mM to about 800 mM.
97. The kit of any one embodiments 78-96, wherein the concentration of the kosmotrope in the equilibration buffer or the wash buffer is about 120 mM to about 600 mM.
98. The kit of any one embodiments 78-97, wherein the concentration of the kosmotrope in the equilibration buffer or the wash buffer is about 120 mM, about 240 mM, about 360 mM, about 480 mM, or about 600 mM.
99. The kit of any one of embodiments 77-94, wherein the concentration of the kosmotrope in the equilibration buffer and/or the wash buffer is about 100 mM to about 800 mM, about 100 mM to about 700 mM, about 100 mM to about 600 mM, about 100 mM to about 500 mM, about
200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM.
100. The kit of any one of embodiments 77-94, wherein the concentration of the kosmotrope in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM.
101. The kit of any one of embodiments 77-100, wherein the kosmotrope is sodium sulfate.
102. The kit of any one of embodiments 77-100, wherein the kosmotrope is potassium phosphate.
103. The kit of any one of embodiments 77-100, wherein the kosmotrope is ammonium sulfate.
104. The kit of any one of embodiments 77-103, wherein the kit further comprises an elution buffer.
105. The kit of embodiment 104, wherein the elution buffer has a lower pH than the equilibration buffer.
106. The kit of embodiment 104 or embodiment 105, wherein the elution buffer has a pH of about 2.0 to about 4.0 or about 2.5 to about 3.0.
107. The kit of any one of embodiments 104-106, wherein the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3.
108. The kit of any one of embodiments 104-106, wherein the elution buffer has a pH of about 2.7, about 2.75, about 2.8, about 2.85, about 2.9, or about 2.95.
109. The kit of embodiment 104, wherein the elution buffer has a higher pH than the equilibration buffer.
110. The kit of embodiment 104 or embodiment 109, wherein the elution buffer has a pH of about 10.0 to about 12.0.
111. The kit of any one of embodiments 104-110, wherein the elution buffer comprises acetic acid.
112. The kit of embodiment 111, wherein the elution buffer comprises about 50 mM acetic acid to about 250 mM acetic acid.
113. The kit of embodiment 111 or embodiment 112, wherein the elution buffer comprises about 150 mM acetic acid at a pH of about 2.8.
114. The kit of any one of embodiments 104-110, wherein the elution buffer comprises sodium acetate, citrate, or glycine.
115. The kit of any one of embodiments 77-114, for use in purifying an antibody or immunoadhesin or a fragment thereof.
116. The kit of any one of embodiments 77-115, wherein the antigen binding protein is a monoclonal antibody.
117. The kit of any one of embodiments 77-116, wherein the antigen binding protein is a human antibody, a chimeric antibody or a humanized antibody.
118. The kit of any one of embodiments 115-117, wherein the antibody is an antibody fragment selected from a Fab, Fab′-SH, Fv, scFv, and (Fab′)2 fragment.
119. The kit of embodiment 118, wherein the antibody fragment is a Fab.
120. The kit of embodiment 119, wherein the Fab comprises a binding site for binding with Protein L that lacks a hydrophobic patch or has a weak hydrophobic patch.
121. The kit of embodiment 120, wherein the binding site for binding with Protein L lacks a hydrophobic patch.
122. The kit of embodiment 120, wherein the binding site for binding with Protein L has a weak hydrophobic patch.
123. The kit of embodiment 120 or embodiment 122, wherein the binding site for binding with Protein L has a weak hydrophobic patch as compared to the binding site for binding with Protein L of a reference antigen binding protein that is monoclonal antibody G6-31.
124. The kit of any one of embodiments 120-123, wherein the binding site for binding with Protein L comprises a hydrophilic patch.
125. The kit of any one of embodiments 77-124, wherein the binding affinity (EC50) and/or the dissociation constant of the antigen binding protein to Protein L has a value that is higher than the value of the binding affinity (EC50) and/or the dissociation constant of a reference antigen binding protein that is monoclonal antibody G6-31 to Protein L.
126. The kit of embodiment 125, wherein the binding affinity (EC50) and/or the dissociation constant of the antigen binding protein to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.
127. The kit of any one of embodiments 115-126, wherein the antibody does not contain an Fc domain.
128. The kit of any one of embodiments 115-127, wherein the antibody is a multispecific antibody.
129. The kit of embodiment 128, wherein the multispecific antibody comprises a VL domain and a second VL domain, and wherein the VL domain is a kappa (κ) VL.
130. The kit of embodiment 129, wherein the VL domain is a κ2 VL.
131. The kit of embodiment 129 or embodiment 130, wherein the VL domain binds poorly to Protein L.
132. The kit of any one of embodiments 129-131, wherein the VL domain binds poorly to Protein L and the second VL domain does not bind to Protein L.
133. The kit of any one of embodiments 129-132, wherein the second VL domain is a lambda (λ) VL.
134. The kit of any one of embodiments 115-127, wherein the antibody comprises a VL domain that is a λ subtype VL or a κ VL.
135. The kit of embodiment 134, wherein the VL domain binds poorly to Protein L.
136. The kit of any one of embodiments 129-135, wherein the VL domain of the antigen binding protein binds poorly to Protein L as compared to a reference antigen binding protein that is monoclonal antibody G6-31.
137. The kit of any one of embodiments 129-136, wherein the VL domain of the antigen binding protein binds weaker to Protein L than a reference antigen binding protein that is monoclonal antibody G6-31.
138. The kit of any one of embodiments 134-137, wherein the VL domain is a κ VL and comprises a modification that weakens binding of the VL domain to Protein L.
139. The kit of embodiment 138, wherein the modification comprises one or more amino acid substitutions in the VL domain.
140. The kit of any one of embodiments 134-139, wherein the VL domain is a κ2 VL.
141. The kit of any one of embodiments 77-140, wherein the antigen binding protein comprises a binding site for binding with Protein L that lacks a hydrophobic patch or has a weak hydrophobic patch.
142. The kit of embodiment 141, wherein the binding site for binding with Protein L lacks a hydrophobic patch.
143. The kit of embodiment 141, wherein the binding site for binding with Protein L has a weak hydrophobic patch.
144. The kit of embodiment 141 or embodiment 143, wherein the binding site for binding with Protein L has a weak hydrophobic patch as compared to the binding site for binding with Protein L of a reference antigen binding protein that is monoclonal antibody G6-31.
145. The kit of any one of embodiments 141-144, wherein the binding site for binding with Protein L comprises a hydrophilic patch.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1. Protein L Chromatography of an Antibody Fragment Using a Kosmotrope

While developing a purification process for a pipeline molecule referred to herein as Fab 1 (a Fab antibody fragment), Protein L was used because this Fab molecule lacks an Fc region, the binding site required for use of Protein A.

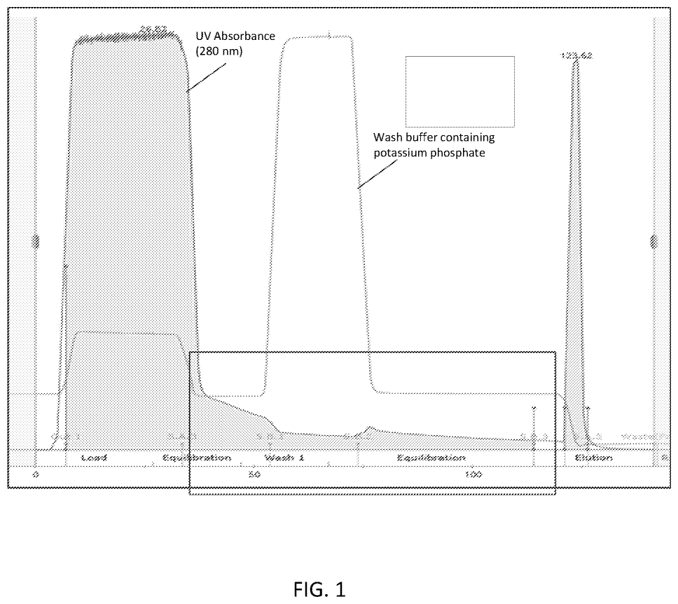

In developing a Protein L chromatography run (FIG. 1), an elevated UV absorbance measured during the wash phases following load and preceding elution was observed. In addition, the UV reading decreased during a phase containing potassium phosphate (a noncorrosive buffering salt used to remove ionic impurities such as host cell DNA), but after this wash phase ended the UV baseline increased again. This result suggested that protein was sloughing off the column following load phase, but the Inventors discovered that this exodus of protein was interrupted and suppressed temporarily by the presence of phosphate, a kosmotrope.

To investigate the use of kosmotropes to enhance Protein L chromatography, a number of studies were conducted to purify Fab 1 by Protein L chromatography using wash buffers supplemented with different concentrations of potassium phosphate (a kosmotrope), sodium sulfate (a stronger kosmotrope), and sodium chloride (a neutral salt with little or no kosmotropic properties). Host cell culture fluid (HCCF) containing the Fab 1 antibody fragment was loaded onto a 7 ml Capto L column at a density of 3.74 g/L and a flow rate of 2.23 ml/min. The column was washed with equilibration buffer (25 mM Tris, 25 mM NaCl, pH 7.7) containing the kosmotrope, (sodium sulphate or potassium phosphate) or with NaCl at concentrations of 600 mM, 480 mM, 360 mM, 240 mM, or 120 mM. Equilibration buffer without a kosmotrope or additional NaCl was used as a negative control. Antibodies were then eluted from the column using 100 mM acetic acid, pH 2.9. Protein concentration as measured by A280, conductivity and pH were monitored throughout the chromatography runs. Chromatography runs were analyzed for yield and quality using SEC-HPLC (TSKGel 2000SW column). As demonstrated below, processes using kosmotropic salts increased yield and purity.

### Study 1: Sodium Sulfate (Strong Kosmotrope)

Six purification runs in which the equilibration buffer used five different concentrations of sodium sulfate to evaluate loss of antibody from the column. Host cell culture fluid (HCCF) containing the Fab 1 antibody fragment was loaded onto a 7 ml Capto L column at a density of 3.74 g/L and a flow rate of 2.23 ml/min. The column was washed with equilibration buffer (25 mM Tris, 25 mM NaCl, pH 7.7) containing sodium sulfate (600 mM, 480 mM, 360 mM, 240 mM, 120 mM), or no sulfate (0 mM; as a negative control). Antibodies were then eluted from the column using 100 mM acetic acid, pH 2.9. Protein concentration as measured by A280, conductivity and pH were monitored throughout the chromatography runs.

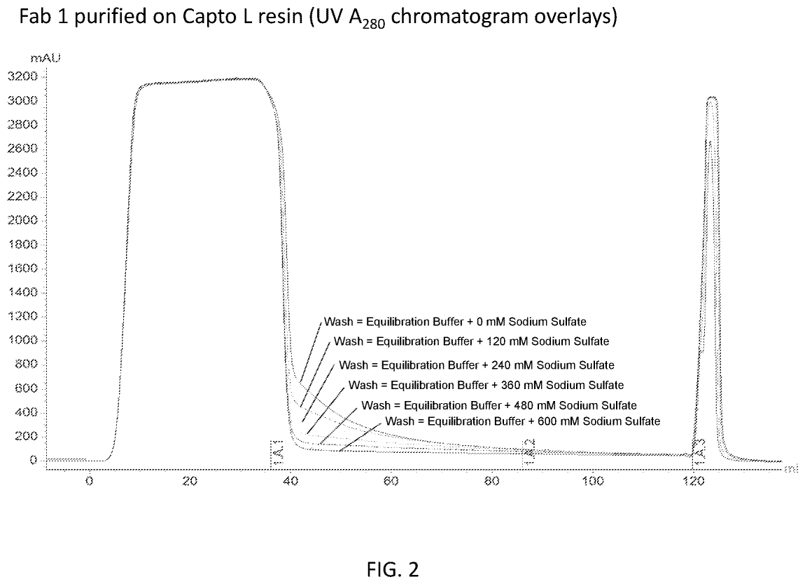

Inspection of the chromatograms (FIG. 2) revealed that higher sulfate concentrations correlated with less protein exiting the column during wash phase, and more protein being recovered during the elution phase.

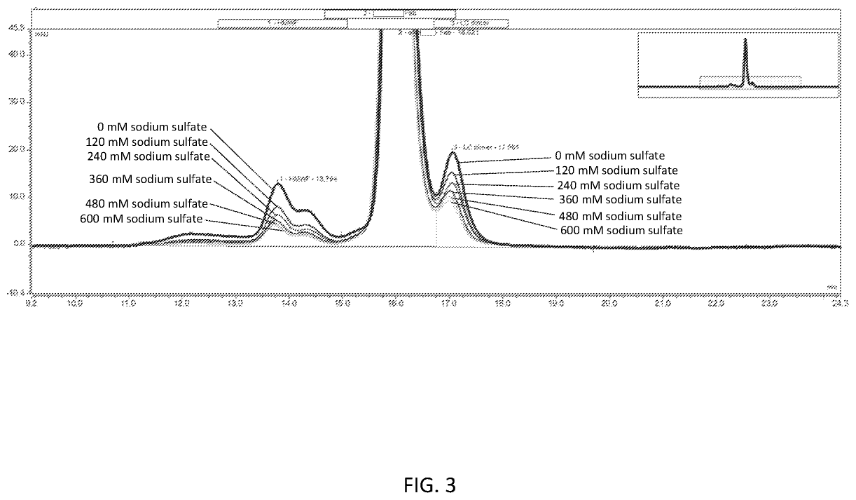

Pools recovered from the elution phase were collected and analyzed by concentration and size-exclusion HPLC (FIG. 3). As shown in Table 2, yield was improved as well as purity when the kosmotrope was included in the wash buffer. Without being bound by theory, this outcome suggests that the kosmotrope is strengthening the binding of main species to Protein L.

It is likely that impurities such as HMWF and LC-dimers may bind more tightly to Protein L than the main species due to having 2 or more binding sites, which can result in worse yield and worse quality for the main species.

TABLE 2
Calculated yield and purity from analysis of eluted pools.

| | Calculated | Main Peak by |
|---|---|---|
| Wash Buffer Additive | Yield (%) | SEC-HPLC (%) |
| 0 mM Sodium Sulfate | 22.71 | 80.28 |
| 120 mM Sodium Sulfate | 32.48 | 85.47 |
| 240 mM Sodium Sulfate | 39.20 | 87.27 |
| 360 mM Sodium Sulfate | 46.37 | 88.89 |
| 480 mM Sodium Sulfate | 52.73 | 90.21 |
| 600 mM Sodium Sulfate | 58.60 | 91.02 |

### Study 2: Potassium Phosphate (Kosmotrope)

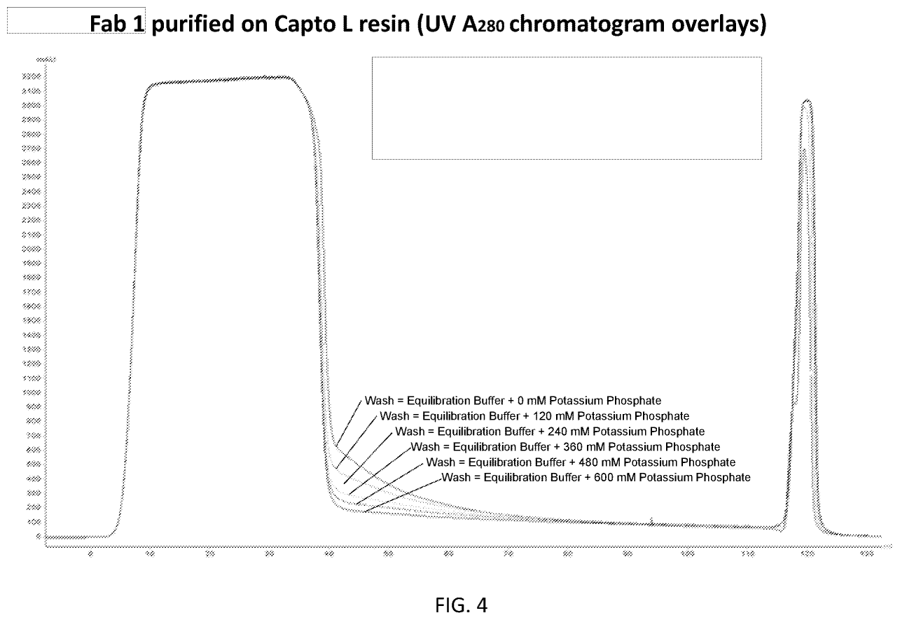

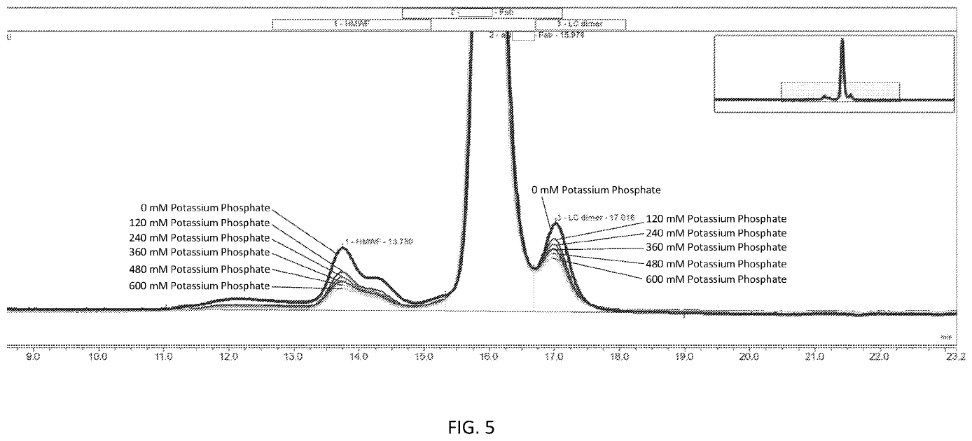

In this study, a different salt with shared kosmotropic nature was evaluated. Six purification runs in which the equilibration buffer used five different concentrations of potassium sulfate (600 mM, 480 mM, 360 mM, 240 mM, 120 mM), or no sulfate (0 mM; as a negative control) were performed. Other chromatography parameters were the same as the sodium sulfate runs. As shown in FIG. 4 and Table 3, a similar outcome although the effect was not a pronounced in the prior study. In the chromatograms (FIG. 4), UV absorbance signal in the post-load wash was increasingly suppressed by increasing concentrations of potassium phosphate. Yield and purity likewise were also improved with increasing concentrations of potassium phosphate (FIG. 5, Table 3), although not as much as with equivalent concentrations of sodium sulfate.

TABLE 3
Calculated yield and purity from analysis of eluted pools.

| | Calculated | Main Peak by |
|---|---|---|
| Wash Buffer Additive | Yield (%) | SEC-HPLC (%) |
| 0 mM Potassium Phosphate | 21.96 | 79.38 |
| 120 mM Potassium Phosphate | 33.05 | 83.55 |
| 240 mM Potassium Phosphate | 36.87 | 85.29 |
| 360 mM Potassium Phosphate | 41.09 | 86.36 |
| 480 mM Potassium Phosphate | 45.45 | 87.50 |
| 600 mM Potassium Phosphate | 50.38 | 88.51 |

### Study 3: Sodium Chloride (Neutral Salt, Control)

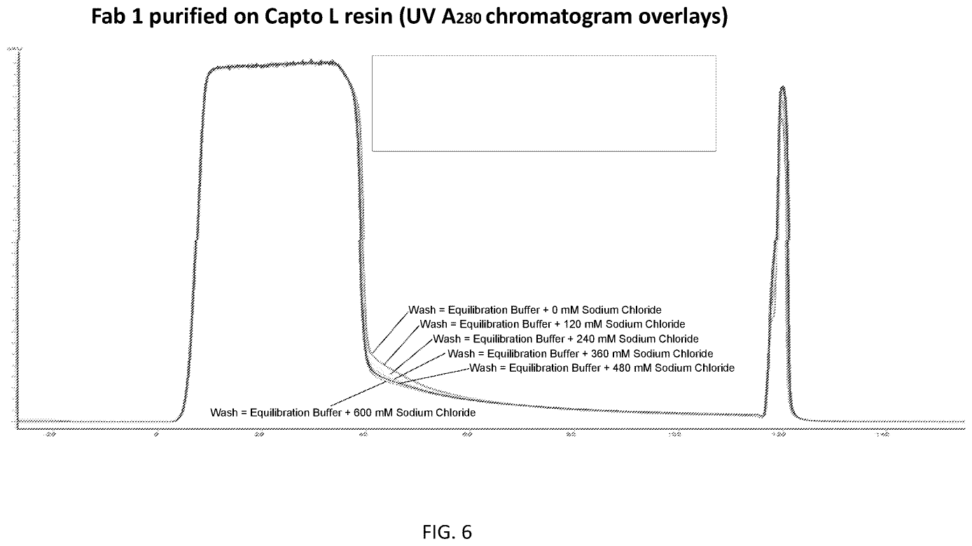

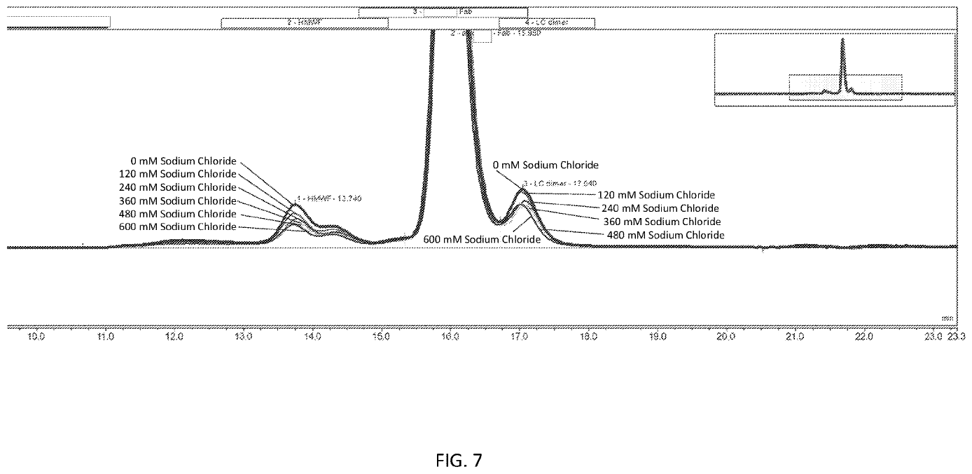

Adding Sodium Chloride to the wash buffer had a minimal effect on rescuing performance of this step. In comparing the chromatograms (FIG. 6), UV absorbance post-wash remained high even at high concentrations of Sodium Chloride, and this was accompanied by a much more modest improvement in yield and purity over the course of the study (FIG. 7, Table 4) than at equivalent concentrations of kosmotropic salts.

TABLE 4
Calculated Yield and Purity from analysis of eluted pools.

| | Calculated | Main Peak by |
|---|---|---|
| Wash Buffer Additive | Yield (%) | SEC-HPLC (%) |
| 0 mM Sodium Chloride | 21.91 | 78.86 |
| 120 mM Sodium Chloride | 26.41 | 81.09 |
| 240 mM Sodium Chloride | 28.60 | 83.70 |
| 360 mM Sodium Chloride | 30.79 | 84.48 |
| 480 mM Sodium Chloride | 32.15 | 84.19 |
| 600 mM Sodium Chloride | 33.43 | 84.88 |

### Example 2. Protein L Chromatography of a Bispecific Antibody Using a Kosmotrope

Poor binding of antibody to Protein L in another molecule, a bispecific antibody bearing one light chain against target antigen A (“anti-A light chain”) and one light chain against target antigen B (“anti-B light chain”), herein referred to as bispecific antibody 1, was observed. The anti-B light chain was designed to not to bind to Protein L by engineering an S12P in the VL region, leaving only the anti-A light chain to serve as the “anchor” to Protein L. The anti-A LC has a VK2 kappa framework, which is a poor binder of Protein L.

To investigate the use of kosmotropes to enhance Protein L chromatography of bispecific antibody 1, chromatography runs were performed in the absence or presence of a kosmotrope. Host cell culture fluid (HCCF) containing the bispecific antibody 1 fragment was filtered using a 0.22μ and diluted in equilibration buffer (25 mM Tris, 25 mM NaCl, pH 7.7) to 0.345 g/L and loaded onto a 1 ml HiTrap Protein L column at a flow rate of 0.4 ml/min. The column was washed with equilibration buffer without a kosmotrope or with 600 mM sodium sulfate. Antibodies were then eluted from the column using 170 mM acetic acid, pH 2.75. Protein concentration as measured by A280, conductivity and pH were monitored throughout the chromatography runs.

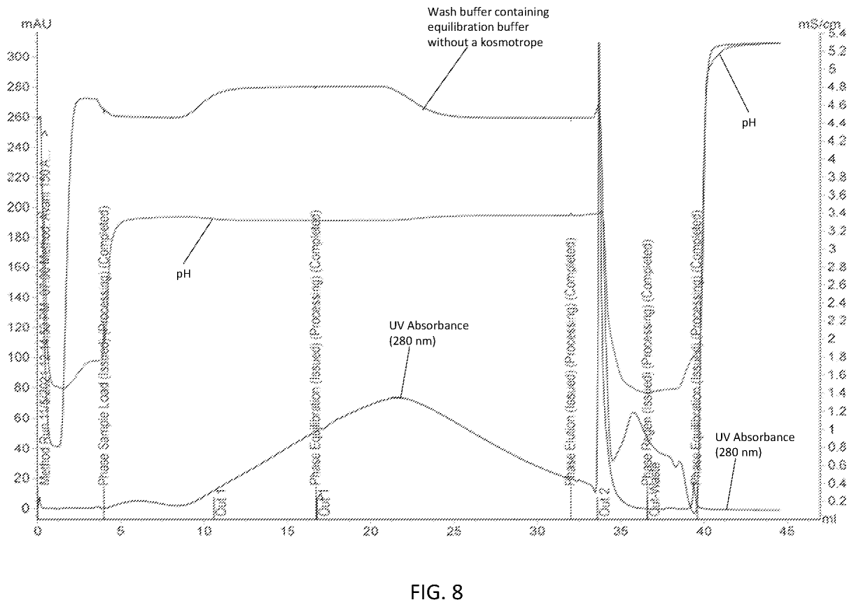

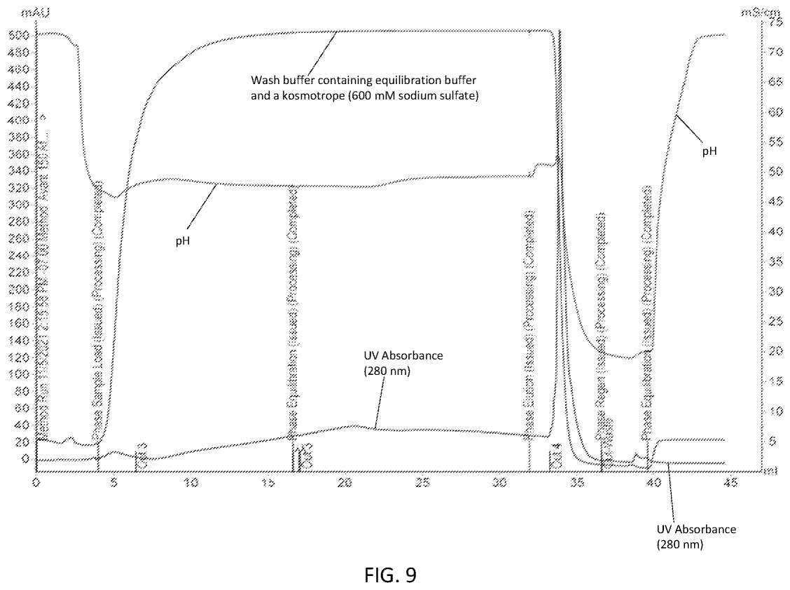

Results of a chromatography run where the wash buffer did not contain a kosmotrope is shown in FIG. 8 and results of a chromatography run where the wash buffer contained a kosmotrope (600 mM sodium sulfate) is shown in FIG. 9. Protein breakthrough and wash-off was decreased in the sulfate-containing experiment resulting in retention of the protein and recovery of a larger elution peak with sulfate than without. Compare FIG. 9 with FIG. 8.

The step yield for the non-sulfate run was calculated to be 10.11%, but in the experiment with Sulfate it was calculated to be 31.49%, a tripling of yield.

What is claimed is:

1. A method for purifying an antigen binding protein comprising a VL domain, the method comprising:
a) binding the antigen binding protein to a Protein L chromatography material,
b) washing the Protein L chromatography material with a wash buffer, wherein the wash buffer comprises an equilibration buffer and a kosmotrope, and
c) eluting the antigen binding protein from the Protein L chromatography material with an elution buffer.

2. A method for improving Protein L purification of an antigen binding protein comprising a VL domain, the method comprising:
a) binding the antigen binding protein to a Protein L chromatography material,
b) washing the Protein L chromatography material with a wash buffer, wherein the wash buffer comprises an equilibration buffer and a kosmotrope, and
c) eluting the antigen binding protein from the Protein L chromatography material with an elution buffer.

3. The method of claim 2, wherein the yield and/or purity of the antigen binding protein is increased compared to Protein L chromatography where the wash buffer does not comprise a kosmotrope.

4. The method of any one of claims 1-3, wherein the equilibration buffer comprises Tris, MES, MOPS, or EDTA.

5. The method of any one of claims 1-4, wherein the equilibration buffer comprises Tris and NaCl.

6. The method of claim 4 or claim 5, wherein the Tris is present in the equilibration buffer at a concentration of about 10 mM to about 50 mM, or about 25 mM.

7. The method of claim 5 or claim 6, wherein the NaCl is present in the equilibration buffer at a concentration of about 10 mM to about 50 mM, or about 25 mM.

8. The method of any one of claims 1-7, wherein the equilibration buffer has a pH of about 4 to about 10.

9. The method of any one of claims 1-8, wherein the equilibration buffer has a pH of about 6.5 to about 8.5, or about 7.7.

10. The method of any one of claims 1-9, wherein the equilibration buffer has a pH of about 7 to about 8.

11. The method of any one of claims 1-10, wherein the wash buffer has a pH of about 4 to about 10.

12. The method of any one of claims 1-11, wherein the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5.

13. The method of any one of claims 1-12, wherein the wash buffer has a pH of about 4, about 4.5, about 5, about 5.5, about 6, about 6.5, about 7, about 7.5, about 8, about 8.5, about 9, about 9.5, or about 10.

14. The method of any one of claims 1-12, wherein the wash buffer has a pH of about 7.0, about 7.1, about 7.2, about 7.3, about 7.4, about 7.5, about 7.6, about 7.7, about 7.8, about 7.9, or about 8.0.

15. The method of any one of claims 1-14, wherein the antigen binding protein is loaded onto the protein L chromatography material in step a) as a host cell culture supernatant.

16. The method of any one of claims 1-14, wherein the antigen binding protein is loaded onto the protein L chromatography material in step a) as a purified polypeptide solution.

17. The method of any one of claims 1-14, wherein the antigen binding protein is loaded onto the protein L chromatography material in step a) as a mixture from a prior purification step.

18. The method of any one of claims 1-14, wherein the antigen binding protein is formulated in equilibration buffer prior to loading onto the protein L chromatography material in step a).

19. The method of any one of claims 1-14 and 18, wherein the antigen binding protein is formulated in equilibration buffer and the kosmotrope prior to loading onto the protein L chromatography material in step a).

20. The method of any one of claims 1-19, wherein the kosmotrope is a phosphate salt or a sulfate salt.

21. The method of any one of claims 1-20, wherein the kosmotrope is potassium phosphate, sodium sulfate, or ammonium sulfate.

22. The method of any one of claims 1-21, wherein the concentration of the kosmotrope in the equilibration buffer or the wash buffer is about 100 mM to about 800 mM.

23. The method of any one of claims 1-21, wherein the concentration of the kosmotrope in the equilibration buffer and the wash buffer is about 100 mM to about 800 mM.

24. The method of any one of claims 1-23, wherein the concentration of the kosmotrope in the equilibration buffer or the wash buffer is about 120 mM to about 600 mM.

25. The method of any one of claims 1-24, wherein the concentration of the kosmotrope in the equilibration buffer or the wash buffer is about 120 mM, about 240 mM, about 360 mM, about 480 mM, or about 600 mM.

26. The method of any one of claims 1-21, wherein the concentration of the kosmotrope in the equilibration buffer and/or the wash buffer is about 100 mM to about 800 mM, about 100 mM to about 700 mM, about 100 mM to about 600 mM, about 100 mM to about 500 mM, about 200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM.

27. The method of any one of claims 1-21, wherein the concentration of the kosmotrope in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM.

28. The method of any one of claims 1-27, wherein the kosmotrope is sodium sulfate.

29. The method of any one of claims 1-27, wherein the kosmotrope is potassium phosphate.

30. The method of any one of claims 1-27, wherein the kosmotrope is ammonium sulfate.

31. The method of any one of claims 1-30, wherein the elution buffer has a lower pH than the equilibration buffer.

32. The method of any one of claims 1-31, wherein the elution buffer has a pH of about 2.0 to about 4.0, or about 2.5 to about 3.0.

33. The method of any one of claims 1-32, wherein the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3.

34. The method of any one of claims 1-32, wherein the elution buffer has a pH of about 2.7, about 2.75, about 2.8, about 2.85, about 2.9, or about 2.95.

35. The method of any one of claims 1-30, wherein the elution buffer has a higher pH than the equilibration buffer.

36. The method of claim 35, wherein the elution buffer has a pH of about 10.0 to about 12.0.

37. The method of any one of claims 1-36, wherein the elution buffer comprises acetic acid.

38. The method of claim 37, wherein the elution buffer comprises about 50 mM acetic acid to about 250 mM acetic acid.

39. The method of claim 37 or claim 38, wherein the elution buffer comprises about 150 mM acetic acid at a pH of about 2.8.

40. The method of any one of claims 1-36, wherein the elution buffer comprises sodium acetate, citrate, or glycine.

41. The method of any one of claims 1-40, wherein the antigen binding protein binds poorly to Protein L.

42. The method of any one of claims 1-41, wherein the antigen binding protein binds poorly to Protein L as compared to a reference antigen binding protein that is monoclonal antibody G6-31.

43. The method of any one of claims 1-41, wherein the antigen binding protein binds weaker to Protein L than a reference antigen binding protein that is monoclonal antibody G6-31.

44. The method of any one of claims 1-43, wherein the antigen binding protein is an antibody or immunoadhesin or a fragment thereof.

45. The method of any one of claims 1-44, wherein the antigen binding protein is a monoclonal antibody.

46. The method of any one of claims 1-45, wherein the antigen binding protein is a human antibody, a chimeric antibody or a humanized antibody.

47. The method of any one of claims 44-46, wherein the antibody is an antibody fragment selected from a Fab, Fab′-SH, Fv, scFv, and (Fab′)2 fragment.

48. The method of claim 47, wherein the antibody fragment is a Fab.

49. The method of claim 48, wherein the Fab comprises a binding site for binding with Protein L that lacks a hydrophobic patch or has a weak hydrophobic patch.

50. The method of claim 49, wherein the binding site for binding with Protein L lacks a hydrophobic patch.

51. The method of claim 49, wherein the binding site for binding with Protein L has a weak hydrophobic patch.

52. The method of any one of claims 49-51, wherein the binding site for binding with Protein L has a weak hydrophobic patch as compared to the binding site for binding with Protein L of a reference antigen binding protein that is monoclonal antibody G6-31.

53. The method of any one of claims 48-52, wherein the binding site for binding with Protein L comprises a hydrophilic patch.

54. The method of any one of claims 1-53, wherein the binding affinity (EC50) and/or the dissociation constant of the antigen binding protein to Protein L has a value that is higher than the value of the binding affinity (EC50) and/or the dissociation constant of a reference antigen binding protein that is monoclonal antibody G6-31 to Protein L.

55. The method of claim 54, wherein the binding affinity (EC50) and/or the dissociation constant of the antigen binding protein to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.

56. The method of any one of claims 44-55, wherein the antibody does not contain an Fc domain.

57. The method of any one of claims 44-46, wherein the antibody is a multispecific antibody.

58. The method of claim 57, wherein the multispecific antibody comprises the VL domain and a second VL domain, and wherein the VL domain is a kappa (κ) VL.

59. The method of claim 58, wherein the VL domain is a κ2 VL.

60. The method of any one of claims 57-59, wherein the VL domain binds poorly to Protein L.

61. The method of any one of claims 58-60, wherein the VL domain binds poorly to Protein L and the second VL domain does not bind to Protein L.

62. The method of any one of claims 58-61, wherein the second VL domain is a lambda (λ) VL.

63. The method of any one of claims 1-56, wherein the VL domain is a λ subtype VL or a κ VL.

64. The method of claim 63, wherein the VL domain binds poorly to Protein L.

65. The method of any one of claims 60-64, wherein the VL domain binds poorly to Protein L as compared to the binding of the VL domain of a reference antigen binding protein that is monoclonal antibody G6-31 to Protein L.

66. The method of any one of claims 63-65, wherein the VL domain is a κ VL and comprises a modification that weakens binding of the VL domain to Protein L.

67. The method of claim 66, wherein the modification comprises one or more amino acid substitutions in the VL domain.

68. The method of any one of claims 63-67, wherein the VL domain is a κ2 VL.

69. The method of any one of claims 1-48 and 56-68, wherein the antigen binding protein comprises a binding site for binding with Protein L that lacks a hydrophobic patch or has a weak hydrophobic patch.

70. The method of claim 69, wherein the binding site for binding with Protein L lacks a hydrophobic patch.

71. The method of claim 69, wherein the binding site for binding with Protein L has a weak hydrophobic patch.

72. The method of any one of claims 69-71, wherein the binding site for binding with Protein L has a weak hydrophobic patch as compared to the binding site for binding with Protein L of a reference antigen binding protein that is monoclonal antibody G6-31.

73. The method of any one of claims 69-71, wherein the binding site for binding with Protein L comprises a hydrophilic patch.

74. The method of any one of claims 1-73, wherein the protein L chromatography material is a Pierce™ Protein L chromatography cartridge, a Capto™ L chromatography, HiTrap® Protein L chromatography, a KanCap™ L chromatography, a TOYOPEARL® AF-rProtein L-650F chromatography, or a MabSelect™ VL chromatography.

75. A composition comprising an antigen binding protein purified by a method comprising the method of any one of claims 1-74.

76. The composition of claim 75, wherein the composition comprises one or more pharmaceutical excipients.

77. A kit for purifying an antigen binding protein comprising a VL domain, wherein the kit comprises a Protein L chromatography material and a kosmotrope.

78. The kit of claim 77, wherein the kit further comprises an equilibration buffer.

79. The kit of claim 78, wherein the equilibration buffer comprises Tris, MES, MOPS, or EDTA.

80. The kit of claim 78 or claim 79, wherein the equilibration buffer comprises Tris and NaCl.

81. The kit of claim 79 or claim 80, wherein the Tris is present in the equilibration buffer at a concentration of about 10 mM to about 50 mM or about 25 mM.

82. The kit of claim 80 or claim 81, wherein the NaCl is present in the equilibration buffer at a concentration of about 10 mM to about 50 mM or about 25 mM.

83. The kit of any one of claims 78-82, wherein the equilibration buffer has a pH of about 4 to about 10.

84. The kit of any one of claims 78-83, wherein the equilibration buffer has a pH of about 6.5 to about 8.5, or about 7.7.

85. The kit of any one of claims 78-84, wherein the equilibration buffer has a pH of about 7 to about 8.

86. The kit of any one of claims 77-85, wherein the kit further comprises a wash buffer.

87. The kit of claim 86, wherein the wash buffer has a pH of about 4 to about 10.

88. The kit of claim 86 or claim 87, wherein the wash buffer has a pH of about 4 to about 10, about 4 to about 9.5, about 4 to about 9, about 4 to about 8.5, about 4 to about 8, about 4 to about 7.5, about 5 to about 10, about 5 to about 9.5, about 5 to about 9, about 5 to about 8.5, about 5 to about 8, about 5 to about 7.5, about 6 to about 10, about 6 to about 9.5, about 6 to about 9, about 6 to about 8.5, about 6 to about 8, or about 6 to about 7.5.

89. The kit of any one of claims 86-88, wherein the wash buffer has a pH of about 4, about 4.5, about 5, about 5.5, about 6, about 6.5, about 7, about 7.5, about 8, about 8.5, about 9, about 9.5, or about 10.

90. The kit of any one of claims 86-88, wherein the wash buffer has a pH of about 7.0, about 7.1, about 7.2, about 7.3, about 7.4, about 7.5, about 7.6, about 7.7, about 7.8, about 7.9, or about 8.0.

91. The kit of any one of claims 86-90, wherein the wash buffer comprises the equilibration buffer and the kosmotrope.

92. The kit of any one of claims 78-91, wherein the equilibration buffer further comprises the kosmotrope.

93. The kit of any one of claims 77-92, wherein the kosmotrope is a phosphate salt or a sulfate salt.

94. The kit of any one of claims 77-93, wherein the kosmotrope is potassium phosphate, sodium sulfate, or ammonium sulfate.

95. The kit of any one claims 78-94, wherein the concentration of the kosmotrope in the equilibration buffer or the wash buffer is about 100 mM to about 800 mM.

96. The kit of any one of claims 78-94, wherein the concentration of the kosmotrope in the equilibration buffer and the wash buffer is about 100 mM to about 800 mM.

97. The kit of any one claims 78-96, wherein the concentration of the kosmotrope in the equilibration buffer or the wash buffer is about 120 mM to about 600 mM.

98. The kit of any one claims 78-97, wherein the concentration of the kosmotrope in the equilibration buffer or the wash buffer is about 120 mM, about 240 mM, about 360 mM, about 480 mM, or about 600 mM.

99. The kit of any one of claims 77-94, wherein the concentration of the kosmotrope in the equilibration buffer and/or the wash buffer is about 100 mM to about 800 mM, about 100 mM to about 700 mM, about 100 mM to about 600 mM, about 100 mM to about 500 mM, about 200 mM to about 800 mM, about 200 mM to about 700 mM, about 200 mM to about 600 mM, about 200 mM to about 500 mM, about 300 mM to about 800 mM, about 300 mM to about 700 mM, about 300 mM to about 600 mM, or about 300 mM to about 500 mM.

100. The kit of any one of claims 77-94, wherein the concentration of the kosmotrope in the equilibration buffer and/or the wash buffer is about 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 mM, 575 mM, 600 mM, 625 mM, 650 mM, 675 mM, 700 mM, 725 mM, 750 mM, 775 mM, or 800 mM.

101. The kit of any one of claims 77-100, wherein the kosmotrope is sodium sulfate.

102. The kit of any one of claims 77-100, wherein the kosmotrope is potassium phosphate.

103. The kit of any one of claims 77-100, wherein the kosmotrope is ammonium sulfate.

104. The kit of any one of claims 77-103, wherein the kit further comprises an elution buffer.

105. The kit of claim 104, wherein the elution buffer has a lower pH than the equilibration buffer.

106. The kit of claim 104 or claim 105, wherein the elution buffer has a pH of about 2.0 to about 4.0 or about 2.5 to about 3.0.

107. The kit of any one of claims 104-106, wherein the elution buffer has a pH of about 2.5, about 2.55, about 2.6, about 2.65, about 2.7, about 2.75, about 2.8, about 2.85, about 2.9, about 2.95, about 3.0, about 3.05, about 3.1, about 3.15, about 3.2, about 3.25, or about 3.3.

108. The kit of any one of claims 104-106, wherein the elution buffer has a pH of about 2.7, about 2.75, about 2.8, about 2.85, about 2.9, or about 2.95.

109. The kit of claim 104, wherein the elution buffer has a higher pH than the equilibration buffer.

110. The kit of claim 104 or claim 109, wherein the elution buffer has a pH of about 10.0 to about 12.0.

111. The kit of any one of claims 104-110, wherein the elution buffer comprises acetic acid.

112. The kit of claim 111, wherein the elution buffer comprises about 50 mM acetic acid to about 250 mM acetic acid.

113. The kit of claim 111 or claim 112, wherein the elution buffer comprises about 150 mM acetic acid at a pH of about 2.8.

114. The kit of any one of claims 104-110, wherein the elution buffer comprises sodium acetate, citrate, or glycine.

115. The kit of any one of claims 77-114, for use in purifying an antibody or immunoadhesin or a fragment thereof.

116. The kit of any one of claims 77-115, wherein the antigen binding protein is a monoclonal antibody.

117. The kit of any one of claims 77-116, wherein the antigen binding protein is a human antibody, a chimeric antibody or a humanized antibody.

118. The kit of any one of claims 115-117, wherein the antibody is an antibody fragment selected from a Fab, Fab′-SH, Fv, scFv, and (Fab′)2 fragment.

119. The kit of claim 118, wherein the antibody fragment is a Fab.

120. The kit of claim 119, wherein the Fab comprises a binding site for binding with Protein L that lacks a hydrophobic patch or has a weak hydrophobic patch.

121. The kit of claim 120, wherein the binding site for binding with Protein L lacks a hydrophobic patch.

122. The kit of claim 120, wherein the binding site for binding with Protein L has a weak hydrophobic patch.

123. The kit of claim 120 or claim 122, wherein the binding site for binding with Protein L has a weak hydrophobic patch as compared to the binding site for binding with Protein L of a reference antigen binding protein that is monoclonal antibody G6-31.

124. The kit of any one of claims 120-123, wherein the binding site for binding with Protein L comprises a hydrophilic patch.

125. The kit of any one of claims 77-124, wherein the binding affinity (EC50) and/or the dissociation constant of the antigen binding protein to Protein L has a value that is higher than the value of the binding affinity (EC50) and/or the dissociation constant of a reference antigen binding protein that is monoclonal antibody G6-31 to Protein L.

126. The kit of claim 125, wherein the binding affinity (EC50) and/or the dissociation constant of the antigen binding protein to Protein L has a value that is at least 15%, at least 25%, at least 50%, at least 75%, at least 100%, at least 150%, at least 200%, at least 300%, at least 400%, or at least 500% higher than the value of the binding affinity (EC50) and/or the dissociation constant of a reference antigen binding protein that is monoclonal antibody G6-31 to Protein L.

127. The kit of any one of claims 115-126, wherein the antibody does not contain an Fc domain.

128. The kit of any one of claims 115-127, wherein the antibody is a multispecific antibody.

129. The kit of claim 128, wherein the multispecific antibody comprises a VL domain and a second VL domain, and wherein the VL domain is a kappa (κ) VL.

130. The kit of claim 129, wherein the VL domain is a κ2 VL.

131. The kit of claim 129 or claim 130, wherein the VL domain binds poorly to Protein L.

132. The kit of any one of claims 129-131, wherein the VL domain binds poorly to Protein L and the second VL domain does not bind to Protein L.

133. The kit of any one of claims 129-132, wherein the second VL domain is a lambda (λ) VL.

134. The kit of any one of claims 115-127, wherein the antibody comprises a VL domain that is a λ subtype VL or a κ VL.

135. The kit of claim 134, wherein the VL domain binds poorly to Protein L.

136. The kit of any one of claims 129-135, wherein the VL domain of the antigen binding protein binds poorly to Protein L as compared to a reference antigen binding protein that is monoclonal antibody G6-31.

137. The kit of any one of claims 129-136, wherein the VL domain of the antigen binding protein binds weaker to Protein L than a reference antigen binding protein that is monoclonal antibody G6-31.

138. The kit of any one of claims 134-137, wherein the VL domain is a κ VL and comprises a modification that weakens binding of the VL domain to Protein L.

139. The kit of claim 138, wherein the modification comprises one or more amino acid substitutions in the VL domain.

140. The kit of any one of claims 134-139, wherein the VL domain is a κ2 VL.

141. The kit of any one of claims 77-140, wherein the antigen binding protein comprises a binding site for binding with Protein L that lacks a hydrophobic patch or has a weak hydrophobic patch.

142. The kit of claim 141, wherein the binding site for binding with Protein L lacks a hydrophobic patch.

143. The kit of claim 141, wherein the binding site for binding with Protein L has a weak hydrophobic patch.

144. The kit of claim 141 or claim 143, wherein the binding site for binding with Protein L has a weak hydrophobic patch as compared to the binding site for binding with Protein L of a reference antigen binding protein that is monoclonal antibody G6-31.

145. The kit of any one of claims 141-144, wherein the binding site for binding with Protein L comprises a hydrophilic patch.
